(19) United States
(12) Patent Application Publication
(10) Pub. No.: US 2016/0377639 A1
(43) Pub. Date: Dec. 29, 2016

(54) METHODS FOR DETECTING, DIAGNOSING AND TREATING TRAUMATIC BRAIN INJURY

(71) Applicant: William Beaumont Hospital, Royal Oak, MI (US)

(72) Inventor: Ray Bahado-Singh, Grosse Pointe Shores, MI (US)

(21) Appl. No.: 15/191,780

(22) Filed: Jun. 24, 2016

Related U.S. Application Data
(60) Provisional application No. 62/185,603, filed on Jun. 27, 2015.
(60) Provisional application No. 62/186,163, filed on Jun. 29, 2015.

(51) Int. Cl. G01N 33/92; A61B 6/00; A61B 5/055; A61B 6/03; G01N 33/68; G01N 33/50

(52) U.S. Cl. CPC G01N 33/92; G01N 33/6812; G01N 33/50; A61B 5/055; A61B 6/032; A61B 6/037; A61B 6/501; A61B 6/5217; G01N 2560/00; G01N 2800/28; G01N 2800/52

(57) ABSTRACT

The present invention relates to methods for detecting, diagnosing and/or treating traumatic brain injury by detecting in a biological sample from a patient the levels of one or more of the metabolites: methionine sulfoxide, PC aa C 34:4, ATP, AMP, NAD+, ADP, IMP, spermidine, lysoPC a C20:3, C18:1, and proline. In some embodiments, the method also includes diagnosing the patient with traumatic brain injury when the one or more metabolites in the biological sample is at a different level than a statistically validated threshold for the one or more metabolites, and CT, MRI, or PET indicates traumatic brain injury to the brain of the patient. In further embodiments, once traumatic brain injury is diagnosed, the patient is treated for the traumatic brain injury.

### CROSS REFERENCE TO RELATED APPLICATIONS

This application claims priority to U.S. Ser. No. 62/185,603, filed Jun. 27, 2015, and U.S. Ser. No. 62/186,163, filed Jun. 29, 2015. The entire contents of the aforementioned applications are incorporated herein.

### FIELD OF THE INVENTION

The present invention is in the field of biochemistry and medicine and relates to methods for detecting, diagnosing, and/or treating a traumatic brain injury.

### BACKGROUND OF THE INVENTION

Traumatic brain injury (TBI) is a disorder of variable etiology and severity. It is classified as mild (concussion), moderate and severe in intensity. TBI is a major cause of death and morbidity internationally. There are approximately 1.7 million cases in the USA annually. There also are approximately 2.4 million emergency department visits, with nearly one-third of all injury-deaths including a diagnosis of TBI. In addition approximately 5.3 million U.S. residents are living with a disability related to TBI. The total economic cost of TBI is estimated to be 76.5 billion annually. Besides athletes and members of the armed forces, the larger civilian population is at significant TBI risk from motor vehicle crashes, falls, assault, blunt impact and other unknown factors.

The importance of developing objective biomarkers for the detection and diagnosis of TBI cannot be overstated. It is often thought that TBI is associated with dramatic events, and the TBI therefore is unlikely to go undiagnosed. It is true that the identification and treatment of moderate to severe TBI represents an easy diagnostic challenge (compared to mild TBI) because of objective findings and the frequent clarity of the history of the former. But mild TBI is common and its reporting by patients is not. The heavy reliance on patient self-reporting to make the diagnosis of mild TBI is a significant limiting factor since the injury itself can reduce the reliability of the self-report. Further, symptoms such as headache, dizziness, depression and anxiety are frequent among the general population but also are features of TBI, thus confounding the diagnosis of TBI. It is estimated that 50% of concussions in the U.S. (approximately 3.8 million cases) go unreported, highlighting the significance of the challenge of accurate diagnosis.

Mild TBI (or concussion) accounts for up to 90% of TBI cases. It is estimated that currently the majority of concussions, as high as 75%, remain undiagnosed. Screening or diagnostic metabolomics markers would be of great clinical value in high-risk groups that are reluctant or unable to disclose that they had a concussion. Such groups would include amateur or professional athletes, infants and the elderly with impaired mental function. Athletes are at particular risk for recurrent concussions. The long-term consequences of undiagnosed TBI could include brain damage or Chronic Traumatic Encephalopathy (CTE).

Currently, estimates suggest that as many as 60% of TBI cases are undiagnosed. Failure to diagnose TBI is a particular risk for athletes particularly at high school, college and professional levels. These individuals are known to often deny or downplay concussive trauma in order to maintain playing time. A high frequency of failed diagnosis has also been described in the military. The heavy reliance on patient self-reporting to make the diagnosis is a particular challenge in the military when one takes into consideration the loss of consciousness and post-traumatic amnesia, which attend such trauma. The non-specificity and frequency of associated symptoms in the general military population such as depression and anxiety is a further confounder and heightens the need for objective markers for the diagnosis in the military and indeed among all TBI groups.

Given the risk of CTE from repeated concussions, improving diagnostic capabilities is a critical issue. Metabolites can make an important contribution to addressing this challenge. Currently, there is tremendous interest in biomarkers for the determination of the pathogenesis of brain damage after TBI. Biomarkers are needed for determining the severity of injury, the likelihood of late onset deterioration, to predict outcomes, and to guide treatment and monitor efficacy. In spite of significant efforts made over recent decades, there are no identified biomarkers of sufficiently high sensitivity and specificity to achieve clinical utility.

Metabolomics is the newest member of the ‘omics’ high dimensional biological sciences. It identifies and quantifies the concentrations of diverse categories of small (<1500 kd) molecules that form the substrates, product and waste products of the cells metabolism. Metabolite categories include lipids, amino acids, peptides, sugars, nucleic acids, vitamins and metals to name a few. It is estimated that there are up to 100,000 metabolites. The human metabolomic database currently has 15,000 entries. Metabolites are downstream to genes, RNA and proteins and thus metabolomics is believed to give the closest portrayal of the cell phenotype of all the ‘omics’ technologies. Genomics and proteomics are said to suggest a possible mode of operation of the biological system while metabolomics give the actual representation of the system at work. Heralding an exciting age of discovery, metabolomics is being used to explain biological mechanisms, and for biomarker development in common and complex brain disorders such as Parkinsons Disease, amyotropic lateral sclerosis, schizophrenia, and stroke. For example, one or more of the inventors has reported on the development of metabolomic biomarkers for hypoxic ischemic encephalopathy and Alzheimer's Disease.

There is strong preliminary evidence of significant metabolic abnormalities including oxidative stress, mitochondrial dysfunction and excitotoxicity. These manifest with perturbations in lactate, pyruvate, NAD+, NADH, glucose, glutamate and glycerol. One or more of the inventors has also shown inflammation to play a key role in TBI inflammation, and many of the metabolites describe below and pathways such as oxidative stress, ATP and lactate are key markers of inflammation.

### SUMMARY OF THE INVENTION

Metabolomics can be used for the detection of metabolic changes even at micromolar concentrations. Surprisingly, the inventors were able to use this tool to identify metabolic biomarkers of TBI and metabolic pathways that are significantly altered in a mouse model of TBI.

Profound changes in multiple important metabolic pathways were detected in the brain tissue (contralateral hemisphere) of mice subjected to well-validated TBI. Using NMR-based metabolomics on brain tissue, forty-six metabolites were identified. Of these a total of twenty-nine metabolites underwent statistically significant concentration changes in the TBI group. The concentrations of fifteen metabolites were increased in TBI relative to controls. The combination of four metabolites, AMP, followed by NAD+, ADP and IMP in that order appeared to be the most affected metabolites after TBI. Their concentrations were all increased in TBI overall compared to sham-operated controls. The area under the ROC curve was high at 1.0. Stringent permutation testing confirmed that the ROC analysis was statistically significant and not due to chance.

In their analysis of the TBI mice, the inventors also identified biomarkers in the serum of the mice that accurately discriminate TBI cases from sham-operated controls. Significant changes in the concentrations of thirty-six of a total of 150 (24.0%) metabolites were observed after TBI. Correspondingly, there were statistically significant differences in a number of important metabolic pathways in the serum of TBI mice. These include arginine and proline metabolism, glutathione metabolism, cysteine and methionine metabolism and sphingolipid metabolism. A combination of PC aa C34:4 and methionine sulfoxide was found to accurately distinguish TBI from sham-operated mice with an AUC (95% CI)=0.85 (0.664-1.0). Furthermore, a combination of six metabolites: methionine sulfoxide, C3, SM C18:0, SM C18:1, methionine and proline accurately discriminated early from late TBI: AUC (95% CI) 1.0 (1.0-1.0).

In one aspect, disclosed is a method of detecting a level of two or more metabolites in a biological sample, where the method consists of obtaining a biological sample from a human patient, where the biological sample includes two or more of methionine sulfoxide, PC aa C 34:4, ATP, AMP, NAD+, ADP, IMP, spermidine, lysoPC a C20:3, C18:1, and proline; and detecting the level of the two or more metabolites in the biological sample. In some aspects, the sample may be blood serum. In other aspects of the invention, the two or more metabolites may be methionine sulfoxide and PC aa C 34:4; or the two or more metabolites may be methionine sulfoxide, spermidine, lysoPC a C20:3, C18:1, and proline; or the two or more metabolites may be ATP, AMP, ADP, and IMP; or the two or more metabolites may be AMP, NAD+, ADP, and IMP; or the two or more metabolites may be ATP, AMP, NAD+, ADP, and IMP.

In other aspects of the invention, the level of the two or more metabolites may be detected by performing nuclear magnetic resonance (NMR) or mass spectrometry (MS) on the biological sample; or the two or more metabolites may be detected by magnetic resonance spectroscopy (MRS) or proton magnetic resonance spectroscopy (1H-MRS) assessed using magnetic resonance imaging (MRI).

In another aspect, the inventive method is diagnosing traumatic brain injury (TBI) in a human patient, wherein the patient has a brain, and method includes obtaining a biological sample from the human patient, where the biological sample includes one or more metabolites methionine sulfoxide, PC aa C 34:4, ATP, AMP, NAD+, ADP, IMP, spermidine, lysoPC a C20:3, C18:1, and proline; detecting a level of the one or more metabolites in the biological sample; performing computerized tomography (CT) scanning, magnetic resonance imaging (MRI), or positron emission tomography (PET) on the brain of the patient; and diagnosing the patient with traumatic brain injury when (a) the one or more metabolites in the biological sample is at a different level than a statistically validated threshold for the one or more metabolites and (b) the CT, MRI, or PET indicates TBI to the brain of the patient.

In other aspects of the invention, the level of the one or more metabolites may be detected by performing nuclear magnetic resonance (NMR) or mass spectrometry (MS) on the biological sample; or the one or more metabolites may be detected by magnetic resonance spectroscopy (MRS) or proton magnetic resonance spectroscopy (1H-MRS) by using MRI. The sample may be blood serum; the one or more metabolites may be methionine sulfoxide and PC aa C 34:4; the one or more metabolites may be methionine sulfoxide, spermidine, lysoPC a C20:3, C18:1, and proline; the one or more metabolites may be ATP, AMP, ADP, and IMP; the one or more metabolites may be AMP, NAD+, ADP, and IMP; or the one or more metabolites may be ATP, AMP, NAD+, ADP, and IMP.

A further aspect is a method of diagnosing and treating TBI in a subject, the method comprising: obtaining a biological sample from the human subject, where the biological sample includes one or more metabolites methionine sulfoxide, PC aa C 34:4, ATP, AMP, NAD+, ADP, IMP, spermidine, lysoPC a C20:3, C18:1, and proline; detecting a level of the one or more metabolites in the biological sample; diagnosing the subject with traumatic brain injury when the one or more metabolites in the biological sample is at a different level than a statistically validated threshold for the one or more metabolites; and administering a therapeutically effective amount of a treatment for TBI to the diagnosed subject. With the diagnostic and treatment method, the treatment may be side-lining the subject, for example, for about 1-2 hours. In other aspects of this invention, the level of the one or more metabolites may be detected by performing nuclear magnetic resonance (NMR) or mass spectrometry (MS) on the biological sample; or the one or more metabolites may be detected by magnetic resonance spectroscopy (MRS) or proton magnetic resonance spectroscopy (1H-MRS) assessed using MRI. The sample may be blood serum; the one or more metabolites may be methionine sulfoxide and PC aa C 34:4; the one or more metabolites may be methionine sulfoxide, spermidine, lysoPC a C20:3, C18:1, and proline; the one or more metabolites may be ATP, AMP, ADP, and IMP; the one or more metabolites may be AMP, NAD+, ADP, and IMP; or the one or more metabolites may be ATP, AMP, NAD+, ADP, and IMP.

In one embodiment, the present inventive method includes providing medical services for a human patient suspected of having or having TBI, this method including requesting a biological sample from and diagnostic information about the patient, where the diagnostic information is a level of one or more metabolites methionine sulfoxide, PC aa C 34:4, ATP, AMP, NAD+, ADP, IMP, spermidine, lysoPC a C20:3, C18:1, and proline in the biological sample; and administering a therapeutically effective amount of a treatment for TBI when the diagnostic information indicates that the level of the one or more metabolites in the biological sample is at a different level than a statistically validated threshold for the one or more metabolites. With this diagnostic and treatment method, the treatment may be side-lining the subject, for example, for about 1-2 hours. In other aspects of this invention, the level of the one or more metabolites may be detected by performing magnetic resonance imaging (MRI), nuclear magnetic resonance (NMR) or mass spectrometry (MS) on the biological sample; or the one or more metabolites may be detected by magnetic resonance spectroscopy (MRS) or proton magnetic resonance spectroscopy (1H-MRS) assessed using MRI. The sample may be blood serum; the one or more metabolites may be methionine sulfoxide and PC aa C 34:4; the one or more metabolites may be methionine sulfoxide, spermidine, lysoPC a C20:3, C18:1, and proline; the one or more metabolites may be ATP, AMP, ADP, and IMP; the one or more metabolites may be AMP, NAD+, ADP, and IMP; or the one or more metabolites may be ATP, AMP, NAD+, ADP, and IMP.

Another embodiment of the present invention is a method of monitoring treatment for TBI in a human patient, comprising: requesting a first biological sample from and first diagnostic information about the patient, wherein the first diagnostic information is a level of one or more metabolites methionine sulfoxide, PC aa C 34:4, ATP, AMP, NAD+, ADP, IMP, spermidine, lysoPC a C20:3, C18:1, and proline in the first biological sample; administering a therapeutically effective amount of a treatment for TBI to the patient; after administering the therapeutically effective amount of the treatment for TBI to the patient, requesting a second biological sample from and second diagnostic information about the patient, wherein the second diagnostic information is a level of one or more metabolites methionine sulfoxide, PC aa C 34:4, ATP, AMP, NAD+, ADP, IMP, spermidine, lysoPC a C20:3, C18:1, and proline in the second biological sample; and comparing the first diagnostic information and the second diagnostic information to determine whether the level of the one or more metabolites in the first biological sample is at a different level than the level of the one or more metabolites in the second biological sample. In a further embodiment, the sample may be serum. Based on metabolite response after treatment, the risk of developing certain complications can be predicted. Further, the patient's metabolite profile may be performed before treatment and, based on the concentrations of certain metabolites, the likelihood of successful response can be estimated prior to actual therapy.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIG. 1A is a 2D Principal Component Analysis (PCA) plot showing TBI versus Sham-operated Mice (based on brain tissue samples). FIG. 1B is a 3D PCA plot showing TBI versus Sham-operated Mice (based on brain tissue samples).

FIG. 2A is a 2D Partial Least Square Discriminant analysis (PLS-DA) plot showing TBI versus Sham-operated Mice (based on brain tissue samples). FIG. 2B is a 3D PLS-DA plot showing TBI versus Sham-operated Mice (based on brain tissue samples).

FIG. 3 is a Variable Importance in Projection Plot (VIP) plot showing: TBI versus Sham-operated Group (based on brain tissue samples). Permutation test (2000 repeats) for the PLS-DA Model: p-value=0.0235.

FIG. 4 is an ROC plot for distinguishing TBI as a group from sham-operated mice (based on brain tissue samples). [AUC: 1.0 (1.0-1.0) which indicates perfect separation]. Four metabolites were used in the PLS-DA model: ADP, AMP, NAD+, IMP. Permutation test (1000 repeats) for the ROC analysis with PLS-DA model: p-value=0.03.

FIG. 5 is a heatmap of brain tissue metabolites: TBI versus Sham-operated controls.

FIG. 6 is a heatmap of serum metabolites: TBI versus Sham-operated controls.

FIG. 7A is a 2D Principal Component Analysis (PCA) plot showing TBI versus Sham-operated Mice (based on serum samples). FIG. 7B is a 3D PCA plot showing TBI versus Sham-operated Mice (based on serum samples).

FIG. 8A is a 2D Partial Least Square Discriminant analysis (PLS-DA) plot showing TBI versus Sham-operated Mice (based on serum samples). FIG. 8B is a 3D PLS-DA plot showing TBI versus Sham-operated Mice (based on serum samples).

FIG. 9 shows predictive accuracy following cross-validation (based on serum samples).

FIG. 10 is a Variable Importance in Projection Plot (VIP) plot showing: TBI versus Sham-operated Group (based on serum samples). Permutation test (2000 repeats) for the PLS-DA Model: P-value <0.0025.

FIG. 11 shows ROC curves for distinguishing TBI cases from controls (based on serum samples).

FIG. 12 shows ROC curve for distinguishing Early from Late TBI diagnosis of timing of TBI: (based on serum samples). AUC: 1.0 (1.0-1.0) which means perfect separation. Six metabolites were used in the PLS-DA model: methionine sulfoxide, C3, SM C18:0, SM C18:1, methionine, and proline. If the model includes the weight variable, the AUC performance was same as metabolites only (AUC=1.0)

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well-known and commonly employed in the art. Although any methods, devices and materials similar or equivalent to those described herein can be used in the practice or testing of the invention, the inventive methods, devices and materials are now described.

### DEFINITIONS

In this specification and the appended claims, the singular forms “a,” “an” and “the” include plural reference unless the context clearly dictates otherwise.

As used in the application, “administering”, when used in conjunction with a treatment means providing or performing medical services with respect to a subject in need of a treatment. For example, when used when used in conjunction with a therapeutic, administering means to deliver a therapeutic directly into or onto a target tissue or to administer a therapeutic to a subject whereby the therapeutic positively impacts the tissue to which it is targeted. “Administering” a composition may be accomplished by oral administration, injection, infusion, absorption or by any method in combination with other known techniques. “Administering” may include the act of self-administration or administration by another person such as, for example, a healthcare provider or other individual.

As used in the present application, “biological sample” means a specimen or culture obtained from any biological source. Biological samples may be obtained from animals (including humans). For example, biological samples may be obtained from a normal subject, a subject suspected of having a traumatic brain injury, or a subject with traumatic brain injury. Biological samples encompass fluids, solids, tissues, gases, and other material derived from a biological organism (e.g., hair or nails). Exemplary fluids include blood products (e.g., whole blood, serum, or plasma) and other fluids typically found within or produced by an organism, such as, sweat, breath condensate, microdialysate (subarachnoid space), lymph, urine, saliva, tears, cerebrospinal fluid, milk, vitreous fluid, amniotic fluid, bile, ascites fluid, pus, cervical vaginal fluid, and the like. Also included within the meaning of the term “biological sample” is an organ or tissue extract (e.g., brain tissue) and culture fluid in which any cells or tissue preparation from a subject has been incubated.

The terms “diagnosis” or “diagnosing” mean a determination (by one or more individuals) that the cause or nature of a problem, situation, or condition in a subject is TBI, or a confirmation of the diagnosis of the disease that includes alternative TBI diagnostics, other signs and/or symptoms (e.g., based in whole or in part on the level(s) of the one or more TBI-indicating metabolites described herein). A “diagnosis” of TBI may include a test or an assessment of the degree of disease severity (e.g., “mild,” “moderate,” or “severe”), current state of disease progression (e.g., “early”, “middle,” or “late” stages of TBI), or include a comparative assessment to an earlier diagnosis (e.g., the TBI's symptoms are advancing, stable, or in remission). A diagnosis may include a “prognosis,” that is, a future prediction of the progression of TBI, based on the observed disease state (e.g., based in whole or in part on the different level(s) of the one or more TBI-indicating metabolites described herein). A diagnosis or prognosis may be based on one or more biological samples obtained from a subject, and may involve a prediction of disease response to a particular treatment or combination of treatments for TBI.

The term “subject” or “patient” as used herein generally refers to any living organism to and may include, but is not limited to, any human, primate, or non-human mammal in need of diagnosis and/or treatment for a condition, disorder or disease (e.g., traumatic brain injury). A “subject” may or may not be exhibiting the signs, symptoms, or pathology of TBI at any stage of any embodiment.

The term “therapeutically effective amount” refers to the amount of treatment (e.g., of an active agent or pharmaceutical compound or composition) that elicits a biological and/or medicinal response in a patient, subject, tissue, or system that is being sought by a researcher, veterinarian, medical doctor or other clinician, or any combination thereof. A biological or medicinal response may include, for example, one or more of the following: (1) preventing a disorder, disease, or condition in an individual that may be predisposed to the disorder, disease, or condition but does not yet experience or display pathology or symptoms of the disorder, disease, or condition, (2) inhibiting a disorder, disease, or condition in an individual that is experiencing or displaying the pathology or symptoms of the disorder, disease, or condition or arresting further development of the pathology and/or symptoms of the disorder, disease, or condition, and/or (3) ameliorating a disorder, disease, or condition in an individual that is experiencing or exhibiting the pathology or symptoms of the disorder, disease, or condition or reversing the pathology and/or symptoms disorder, disease, or condition experienced or exhibited by the individual.

The term “traumatic brain injury” or “TBI” mean a medical disorder that generally is the result of a sudden, violent blow or jolt to the head of a subject. The subject's brain is launched into a collision course with the inside of the skull, resulting in one or more of the following conditions: bruising or edema (swelling) of the brain, tearing of nerve fibers, subarachnoid and/or intracerebral hemorrhaging, blood clots, subdural hematomas, or necrosis of the brain tissue.

The term “treatment” or “treating” as used herein refers to administrating a medicine or the performance of medical procedures with respect to a subject, for either prophylaxis (prevention) or to cure or reduce the extent of or likelihood of occurrence or recurrence of an infirmity or malady or condition or event in the instance where the subject is afflicted. As related to the present invention, the term may also mean administrating medicine or the performance of medical procedures as therapy, prevention or prophylaxis of traumatic brain injury.

The disruption of energy metabolism is a well-recognized feature of TBI. Microdialysis catherization allows monitoring of energy related small molecules in an ongoing fashion in the extracellular fluid of clinical subjects. Other techniques such as measurement of arteriovenous gradient, oxygen salivation measurements and radiographic techniques such as positron emission tomography (PET) imaging studies using labeled 2-dioxy-D-glucose tracers have provided unique metabolic insights into brain changes in TBI. These reveal profound changes in glucose uptake and metabolism of the brain after TBI. Glucose is the preferred substrate of the brain and is metabolized anaerobically through the glycolytic pathway to pyruvate with the generation of ATP and NADH. This occurs in the cytosol of the cell. Pyruvate is then converted to acetyl CoA which enters the tricarboxyalic acid cycle (TCA) in the mitochondria. The TCA generates electrons used in the inner mitochondrial membrane to create a proton gradient in the inner mitochondrial membrane and ultimately into the synthesis of the ATP, the major energy source of the cell, from ADP.

The early stages of TBI are said to be characterized by hyper-metabolism of glucose. Later on, lactate is said to substitute for glucose as the main energy source. The inventors found significant changes in the concentration of nucleosides (ATP, ADP, and AMP) whose hydrolysis is the major energy source of the cells. The concentration of each was elevated in TBI cases. Energy that is produced from glucose is known to be critically linked to neurotransmission. Inosine monophosphate (IMP) is a precursor of ATP; and the inventors also found a very significant elevation in the concentration of IMP in TBI cases.

Glucose is the most widely used of the monosaccharides in the body, however, the other monosaccharides such as fructose and galactose are important sources of fuel. These other monosaccharides can be metabolized in the glycolytic pathway by conversion to uridine diphosphate glucose (‘UDP-glucose’). UDP-glucose was found to be significantly elevated in TBI cases suggesting the utilization of these other monosaccharides in the metabolism of the traumatized brain. Neurons are now believed to metabolize lactate, previously thought to be only a waste product from the Glycolytic TCA cycle. Low free lactate levels in TBI have been correlated with better long term outcome. In the present study, there was no change in lactate levels noted. Further, the inventors did not have data on outcomes given the design of the study. Lactate/pyruvate ratios have been used to predict outcome in TBI. Pyruvate levels were not measured in this analysis.

The inventors' metabolomics analysis found an increase in glutamate and a decrease in 4-aminobutyrate [common name gamma-aminobutyric acid (GABA)]. Glutamine is the main excitatory neurotransmitter while GABA or 4-amino butyrate is the main inhibitory neurotransmitter. Maintaining the balance between these two agents is critical for normal neurological function. After TBI, the release of glutamine results in a state of excitotoxicity that leads to further neuronal injury, and death and dysfunction of neurons. GABA modulates the excitatory effects of glutamate and is released from interneurons. Prior microdialysis studies in humans revealed significant glutamate perturbations in TBI.

Alanine is known to be important for lymphocyte reproduction and immunity overall. Like GABA, alanine, taurine and glycine, it is also an inhibitory neurotransmitter in the brain. The inventors observed a significant reduction in alanine levels without a statistically significant change in taurine and glycine.

N-acetylaspartate (NAA) is a brain specific metabolite found in abundance yet its specific role remains unclear. It is thought to possibly regulate water homeostasis, supply acetate for myelin biosynthesis and serve as a reservoir for glutamate. Using proton magnetic resonance spectroscopy (1H-MRS), NAA levels were demonstrated to undergo relatively prolonged depression after concussion in athletes. The degree of depression of brain NAA is known to correlate with the severity of TBI and the depression to be transient relatively soon after the insult. NAA has been proposed as an indirect marker of cerebral energy state end brain health and for use as a marker for determining the time of safe return of athletes following concussion on the field. The inventors however observed a significant elevation of NAA levels after TBI. Separate analysis of early- versus late-TBI was not possible because of the small number of cases, and the inventors were not able to follow the temporal changes with TBI. Further, the inventors utilized tissue from the contralateral hemisphere rather than the hemisphere subjected to the trauma.

Pathway analysis revealed a significant disturbance of purine metabolism (see, Table 3). This is not surprising given the energy crisis that has been consistently reported after TBI. This is the pathway by which key energy biomolecules such as AMP, GMP, NAD are synthesized. In addition the purine nucleotides are basic constituents for RNA and DNA synthesis. RNA and DNA synthesis would be judged to be critical to the repair process after the tissue death and damage sustained by the brain. Hypoxanthine, an intermediate of purine catabolism was also found to be elevated in the present study. The alanine, aspartate, glutamine pathway was significantly affected.

Glutathione is an important agent in antioxidant defenses. Oxidative stress is a critical feature of TBI and an important mechanism in the inflammatory cascade that is recognized to attend this injury. After TBI oxidative stress markers are known to increase while antioxidant defense enzymes such as glutathione are found to decrease. The significant changes found in the glutathione pathway are therefore consistent with the established literature.

Branched chain amino acids (isoleucine, leucine, valine) are thought to possibly function in the control of intracranial pressure and cerebral oxygen consumption. Prolonged depression of branched chain amino acids was observed in the jugular veins of severe TBI patients.

Overall, using an NMR-based metabolomics platform, the inventors found significant disturbance in energy metabolism including evidence of profound mitochondrial dysfunction in the contralateral hemisphere of TBI mice. There was in addition disturbances in antioxidant defense, pointing to the role of oxidative stress as a consequence of the injury and additional metabolic changes that could be plausibly linked.

The inventors' brain tissue metabolite analysis provides important and direct information on the impact of TBI on the primary affected organ. However, in the usual clinical context, direct access to brain tissue may be severely limited. Further, the damage in TBI is longitudinal and progressive after the initial trauma; therefore, serial measurement of metabolite levels may be the preferred route to profile ongoing damage, to monitor treatment response, and to make an accurate prognosis. Blood-based biomarkers can be repeatedly accessed with relatively low effort and low cost.

Based on the brain extract data, AMP, NAD+, ADP and IMP were the most discriminating metabolites between TBI and sham controls; while in serum, spermidine, methionine sulfoxide, lysoPC a C20:3, C18:1 and proline were the most powerful predictors of TBI. Mitochondrial dysfunction appeared to be the principal feature of the alterations in brain tissue.

Pathway analysis also confirmed the different effects of TBI on the brain tissue metabolome versus the serum metabolome. In brain tissue, the inventors found that purine, alanine, glutathione, valine and tyrosine pathways were among the metabolic pathways that were most affected. This appeared to be in contrast to what was noted in the serum (Table 8) with glutathione being the only pathway that appeared to be altered in common between the two tissues.

An additional finding of the present study was that a combination of six serum metabolites, sensitively discriminated TBI based on the timing of the injury (methionine sulfoxide, C3, SM C18:0, SM C18:1, methionine, and proline). While the number of cases was small, the signal was unusually strong and appears to be statistically quite significant. The timing of TBI may have clinical value in determining the original onset of trauma. This is particularly important among the elderly in nursing homes and for monitoring the progress of a patient's brain injury and recovery. One practical benefit to monitoring injury progression and resolution could include for example the development of objective standards for determining the appropriate time of return to play for athletes who have suffered concussions. At present this remains an unresolved and a contentious issue.

The Pathway analysis provided important insights into the cellular mechanisms of injury in TBI, especially from the perspective of events taking place in serum. The most significant changes appeared to occur in the arginine and proline metabolic pathway. Arginine decarboxylate (ADC) is a rate limiting enzyme in the decarboxylation of arginine to agmatine. Agmatine is a protective neurotransmitter and exogenous agmatine has a neuroprotective effect in animal models of neurotrauma. In spinal cord injury ADC facilitates the production of brain-derived neurotrophic factors. It also reduces neurodegeneration, and is said to accelerate neuronal recovery. Elevated levels of arginine were noted at 24 hours compared to 4 hours (Table 5).

Glutathione plays a well-recognized role in antioxidant defense mechanisms. The inventors observed a significant alteration of glutathione metabolism in this study. Cysteine con-tributes to glutathione synthesis and increased production of glutathione is induced by increased cysteine levels. This is an important mechanism for the removal of excess cysteine.

Oxidative stress has long been known to be partly responsible for the neuronal damage in TBI. Indeed biomarkers of oxidative stress and antioxidant levels have been found to correlate with trauma severity and also predict neurologic outcomes in humans. The abnormalities of cysteine and glutathione metabolism that the inventors found are consistent with these prior reports.

Methionine sulfoxide is another oxidative stress marker. This amino acid is a byproduct of methionine oxidation and thus increases with oxidative stress. Such oxidants can be generated from activated neutrophils; therefore, methionine sulfoxide can be regarded as a metabolite biomarker of oxidative stress in vivo.

Methionine is a dietary amino acid (an amino acid is the building block of proteins) required for normal growth and development of humans. In addition to being a substrate (component) for protein synthesis, it is an intermediate (byproduct) in the transmethylation biochemical reactions, serving as the major methyl (single carbon atom) group donor. This includes the donation of methyl groups for DNA and RNA intermediates. The addition of a methyl group is an important mechanism of control of gene (function) transcription. Methionine is a methyl (single carbon atom) acceptor from folic acid obtained from the diet and then transfers the carbon to DNA, and RNA. Methionine is also required for synthesis of cysteine. Methionine is the metabolic precursor for cysteine. Only the sulfur atom from methionine is transferred to cysteine; the carbon skeleton of cysteine is donated by serine. Cysteine is important in the chemical pathway that leads to the manufacture of the anti-oxidant, glutathione. Glutathione combats oxygen toxicity (also called oxidative stress) hence the designation ‘anti-oxidant’. Oxidative stress is harmful and an important mechanism for neuronal injury in TBI. In disease states or after trauma, cellular requirements may be altered for methionine, cysteine, and taurine.

The inventors found that methionine sulfoxide was a good predictor of TBI. It was however significantly decreased in TBI versus control cases. Interestingly, the inventors also noted that the concentrations of methionine sulfoxide significantly increased over time after TBI perhaps reflecting increasing post-traumatic oxidative stress in the sub-acute phase of the injury.

The inventors also found a significant disturbance of sphingolipid metabolism. Neural membranes contain significant amounts of lipids, including sphingolipids and phospholipids. They are thought to be precursors of lipids involved in signal transduction. Sphingolipids play an important role in neural cell proliferation and apoptosis and are known to support oxidative stress involved in neurologic disorders such as in head and spinal injuries. Serum sphingolipids may, therefore contribute to oxidative damage in TBI or arise from oxidative damage from TBI.

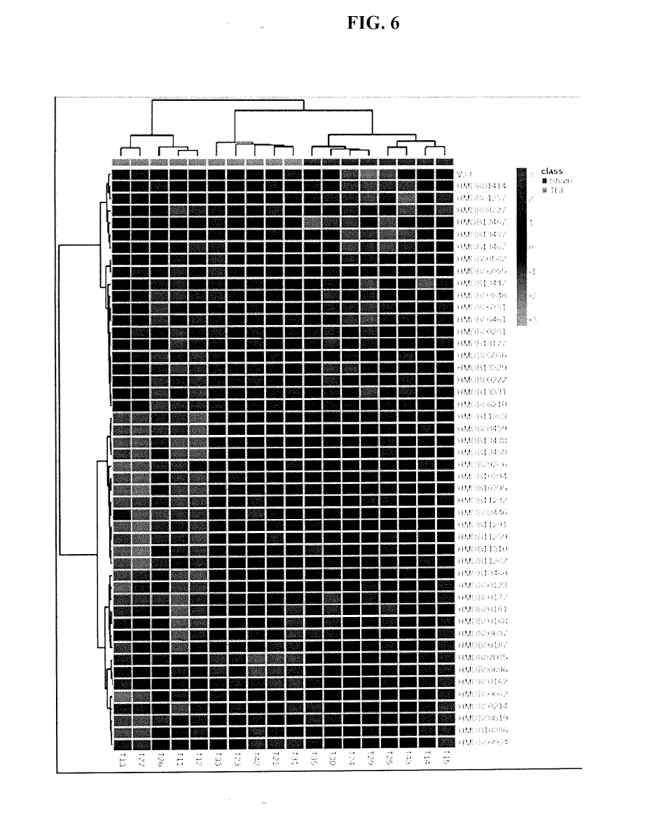

Serum spermidine levels were also significantly elevated in TBI mice versus sham-operated controls, FIG. 6. Spermidine and spermine are cations that play an important role in maintaining DNA structure and function through their roles as immune modulators and antioxidants. Putrescine is a polyamine. Putrescine is related to cadaverine (another polyamine). Both are produced by the breakdown of amino acids in living and dead organisms and both are toxic in high concentrations. Putrescine attacks s-adenosyl methionine and converts it to spermidine. Spermidine in turn attacks another s-adenosyl methionine and converts it to spermine. Putrescine is synthesized in small quantities by healthy living cells. The polyamines, of which putrescine is one of the simplest, appear to be growth factors necessary for cell division. Putrescine apparently has specific role in skin physiology and neuroprotection. Polyamines are required for cell proliferation (e.g. replacement of damaged tissue after disease or injury). Transgenic rodent studies have shown that polyamines play a role in functions such as neuronal protection. Spermidine is a polyamine formed from putrescine. It is found in almost all tissues in association with nucleic acids (building blocks from which DNA and RNA are made). It is thought to help stabilize some membranes and nucleic acid structures.

TBI results in disruption of spermidine-spermine homeostasis leading to production of metabolites that cause ongoing post-traumatic injury. Overall, the present findings further support the role of oxidative stress and antioxidant defenses in propagating and controlling respectively ongoing neuronal damage post brain trauma.

When comparing the brain and serum metabolomes, glutathione metabolism was the only pathway found to be significantly disturbed (p<0.05; fdr<0.05) in both brain and serum following TBI. Glutathione (GSH) is the predominant low molecular weight thiol in all animal cells. Following TBI it has been reported that the levels of GSH are elevated due to the increased activity of gammaglutamylcysteine ethyl ester within the brain. The elevation in GSH concentrations may act as a neuroprotectant against oxidative stress associated with TBI.

Phosphatidylcholine diacyl C 34:4 (PC aa C34:4) is a 1,2-diacyl-sn-glycero-3-phosphocholine in which the acyl groups at C-1 and C-2 contain 34 carbons in total with 4 double bonds. It is a glycerophospholipid in which a phosphorylcholine moiety occupies a glycerol substitution site. Glycerophosphocholines can have different combinations of fatty acids (fatty acids are the constituent subunits of fats) of varying lengths and saturation attached at the C-1 and C-2 positions. These constituent fatty acids are derived from different sources including animal fats, coco butter and sesame oil, while the arachidonic acid moiety is derived from animal fats and eggs. Phospholipids are widespread in nature and are key components of the lipid bilayer of cell membranes, as well as being involved in metabolism and cell signaling. While most phospholipids have a saturated fatty acid on C-1 and an unsaturated fatty acid on C-2 of the glycerol backbone.

Adenosine triphosphate (ATP) consists of a purine base (adenine) which is one of the building blocks for DNA and RNA, attached to the first carbon atom of the sugar molecule, ribose (a pentose sugar). Three phosphate groups are attached at the fifth carbon atom of the ribose. The chemical bonds of the attached phosphate bonds store energy. Energy is transmitted into cells through the uptake of ATP such that ATP is a principal source of energy required for various processes in the cell. These include important cellular processes such as cell movement, for cell division, which is important for cell growth and regeneration. ATP provides the energy for many biochemical processes in the cell. ATP is incorporated into DNA by enzymes called polymerases, during DNA replication and transcription. When ATP is used for the synthesis of DNA, the ribose sugar is converted to deoxyribose. Beyond its role in maintaining energy balance in the cell, ATP is involved in the activation of other important processes e.g. nerve transmission, inflammation and apoptosis i.e. cell death. These are important processes that are disrupted in TBI. ATP also plays a role in cell repair and regeneration and scar tissue formation in response to injury.

As noted above, ATP has 3 phosphate groups that each store potential energy in their chemical bonds. Energy is released for utilization in the many cellular functions as described above. When enzymes called phosphatases (ATPases) break the chemical bond (de-phosphorylation) in the phosphate group the energy is released to fuel cell processes. ATP is thereby converted to ADP which has two phosphate groups.

Further, degradation or cleaving of one of the two remaining phosphate bonds in ADP releases additional energy, resulting in the generation of AMP. Nucleotides are the subunits that are used to form DNA and RNA (ribonucleic acid). AMP is the nucleotide subunit used for RNA synthesis. RNA generation is the first step in the assembly of proteins in gene transcription.

Inosinic acid or inosine monophosphate (IMP) is a nucleoside monophosphate. Nucleotides are organic compounds that consist of a nitrogenous base, a sugar and a phosphate group. They form the structural units or components of DNA and RNA. Nucleosides are nucleotides to which a phosphate has been added. Nucleosides are glycosylamines that can be thought of as nucleotides without a phosphate group. A nucleoside consists simply of a nucleobase (also termed a nitrogenous base) and a 5-carbon sugar (either ribose or deoxyribose), whereas a nucleotide is composed of a nucleobase, a five-carbon sugar, and one or more phosphate groups. Examples of nucleosides include cytidine, uridine, adenosine, guanosine, thymidine and inosine. IMP plays a role in cellular metabolism. It is formed by the deamination or removal of the nitrogen base from AMP.

Nicotinamide adenine dinucleotide (NAD) is a coenzyme found in all living cells. This compound is a dinucleotide, consisting of two nucleotides joined through their phosphate groups. One nucleotide contains an adenine base and the other nicotinamide. Nicotinamide adenine dinucleotide exists in two forms, an oxidized form (NAD+) and a reduced form (NADH). NAD+ is involved in glycolysis, oxidative phosphorylation and the citric acid cycle.

LysoPhosphatidylcholine acyl C20:3 (LysoPC a C20:3) is a lysophospholipid (LyP). It is a monoglycerophospholipid in which a phosphorylcholine chemical group occupies a glycerol substitution site. Lysophosphatidylcholines can have different combinations of fatty acids of varying lengths and saturation attached at the C-1 (sn-1) position. LysoPC(20:3), consists of one chain of mead acid at the C-1 position. The mead acid moiety is derived from fish oils, liver and kidney. Lysophosphatidylcholine is found in small amounts in most tissues. In plasma significant amounts of lysophosphatidylcholine are formed by a specific enzyme system, lecithin:cholesterol acyltransferase (LCAT), which is produced by the liver. The enzyme promotes the transfer of the fatty acids of position sn-2 of phosphatidylcholine to free cholesterol in plasma.

C18:1 is a fatty acid with 18 carbon atoms in the chain and a single double bond within the carbon to carbon chain (—C═C—). Where there are double bonds, one hydrogen atom has been subtracted from the chain. As a result, the maximum possible number of hydrogen atoms are not present in the chain—i.e., the fatty acid is said not to be “saturated” with hydrogen atoms. Thus, the presence of a double bond in the carbon chain makes this an ‘unsaturated’ fatty acid. The C18:1 fatty acid exists in 2 forms, depending on the location of the hydrogen atom relative to the double bond between the carbon atoms. In vaccenic acid, the flanking hydrogen atoms are on opposite sides of the carbon to carbon double bond in space-trans format or isomer. This fatty acid is naturally occurring and can be found in dairy such as milk, cheese and yogurt. In oleic acid the hydrogen atoms are in the cis- or isomer. This fatty acid is found in olive oil, pecan etc.

Proline is an amino acid. Amino acids are the building blocks of proteins. There are a total of 20 amino acids that form the basis of all proteins in the body. Proline is a non-essential amino acid which means that it is synthesized in the body and therefore it is not essential that proline be consumed in the diet. It is therefore considered a ‘non-essential’ amino-acid. Proline is unique from the other 19 amino acids in its chemical structure in that it has an ‘amino’ group (—NH2-) is part of rather than outside its ring structure as is the case with the other amino acids. Amino acids link to each other in chains to form proteins based on the linkage between the amino. This unique chemical structure of proline causes it to play an important role in the folding of the chains of amino acids that are linked together to form proteins and thus determine the shape and ultimately the three dimensional structure of proteins. It is well-recognized that the folding of proteins is critical to their functions.

One aspect of the invention is a method of detecting a level of one or more, two or more, three or more, four or more, five or more, six or more, seven or more, eight or more, nine or more, or ten or more metabolites in a biological sample. This method includes obtaining a biological sample from a human patient, wherein said biological sample has one or more, two or more, three or more, four or more, five or more, six or more, seven or more, eight or more, nine or more, or ten or more of methionine sulfoxide, PC aa C 34:4, ATP, AMP, NAD+, ADP, IMP, spermidine, lysoPC a C20:3, C18:1, and proline; and detecting the level of the one or more, two or more, three or more, four or more, five or more, six or more, seven or more, eight or more, nine or more, or ten or more metabolites in the biological sample.

For example, the methods and assays of the present invention detect one or more metabolites in a biological sample from a subject suspected of having or having a traumatic brain injury. Some metabolites suitable for detection in this invention include: methionine sulfoxide, PC aa C 34:4, ATP, AMP, NAD+, ADP, IMP, spermidine, lysoPC a C20:3, C18:1, and proline, which may be used alone or with other metabolite biomarkers or TBI diagnostics. In serum, spermidine, methionine sulfoxide, lysoPC a C20:3, C18:1, and proline were powerful predictors of TBI; and in brain tissue, AMP, NAD+, ADP and IMP were the most discriminating metabolites.

Also, metabolites suitable for detection in this invention include any of the sixty-five metabolites having statistically significant concentration changes as described in the Examples below, consisting of the twenty-nine metabolites having statistically significant concentration changes as described in Example 2 (Table 2), and the thirty-six metabolites having statistically significant concentration changes as described in Example 4 below (Table 4).

In one embodiment of the invention, each metabolite is considered, evaluated and used individually and separately. In another embodiment of the invention, two metabolites are considered, evaluated and used in combinations of two or more to diagnose TBI. For example, in one aspect of the invention, the metabolites that are detected are PC aa C34:4 and methionine sulfoxide (e.g., in a fluid biological sample, such as, serum). In another aspect of the invention, the metabolites that are detected are ATP, AMP, ADP, and IMP. In another aspect of the invention, the metabolites that are detected are AMP, NAD+, ADP, and IMP. In yet another embodiment, the metabolites that are detected are spermidine, methionine sulfoxide, lysoPC a C20:3, C18:1, and proline (e.g., in a fluid biological sample, such as, serum). In yet another embodiment, the metabolites that are detected are spermidine, methionine sulfoxide, and lysoPC a C20:3 (e.g., in a fluid biological sample, such as, serum).

Further aspects of the invention include detecting any combination of the sixty-five metabolites (as described above and in Examples below) having statistically significant concentration changes, combined in any number and any combination, and diagnosing TBI. In another aspect, the invention includes diagnosing TBI by detecting any combination of the following eleven metabolites in a sample: methionine sulfoxide, PC aa C 34:4, ATP, AMP, NAD+, ADP, IMP, spermidine, lysoPC a C20:3, C18:1, and proline, combined in any number and any combination. For example, using eleven metabolites, all metabolite combinations of 2, 3, 4, 5, 6, 7, 8, 9, 10, and 11 metabolites, combined in any number and any combination, can be used. Thus methionine sulfoxide can be used in any combination with any of the following in combinations of 2-10 other metabolites including: PC aa C 34:4, ATP, AMP, NAD+, ADP, IMP, spermidine, lysoPC a C20:3, C18:1, and proline. Similarly, PC aa C 34:4 can be used in any combination with any of the following in combinations of 2-10 including: methionine sulfoxide, ATP, AMP, NAD+, ADP, IMP, spermidine, lysoPC a C20:3, C18:1, and proline. Similarly, ATP can be used in any combination with any of the following in combinations of 2-10 including methionine sulfoxide, PC aa C 34:4, AMP, NAD+, ADP, IMP, spermidine, lysoPC a C20:3, C18:1, and proline. Similarly, each of AMP, NAD+, ADP, IMP, spermidine, lysoPC a C20:3, C18:1, and proline each can be used in combination with 2-10 of the other metabolites.

One aspect of the inventive is a method for diagnosing traumatic brain injury (TBI) in a human patient, where the patient has a brain and the method includes: obtaining a biological sample from the human patient, wherein said biological sample includes one or more metabolites methionine sulfoxide, PC aa C 34:4, ATP, AMP, NAD+, ADP, IMP, spermidine, lysoPC a C20:3, C18:1, and proline; detecting a level of the one or more metabolites in the biological sample; performing computerized tomography (CT) scanning, magnetic resonance imaging (MRI), or positron emission tomography (PET) on the brain of the patient; and diagnosing the patient with traumatic brain injury when (a) the one or more metabolites in the biological sample is at a different level than a statistically validated threshold for the one or more metabolites and (b) the CT, MRI, or PET indicates TBI to the brain of the patient. As otherwise described herein, the different level may be a reduced level or an elevated level.

Methods of obtaining biological samples from a subject suspected of TBI or having TBI are well known in the art. The biological sample may include one or more metabolites methionine sulfoxide, PC aa C 34:4, ATP, AMP, NAD+, ADP, IMP, spermidine, lysoPC a C20:3, C18:1, and proline. Given the ease and convenience with which appropriate samples for analysis can be collected and analyzed, diagnosis of concussion or mild (moderate or severe) TBI, and ongoing surveillance for progressive deterioration, is potentially feasible.

Any method of measuring or quantitating the amount of metabolite(s) in a biological sample can be used; whether the metabolites are assayed individually, in combination, or by high-throughput methods. Preferred methods are reliable, sensitive and specific for a particular metabolite used as a biomarker in aspects of the present invention. The skilled artisan will recognize which detection methods are appropriate based on the sensitivity of the detection method and the abundance of the target metabolite. Depending on the sensitivity of the detection method and the abundance of the target metabolite, amplification may or may not be required prior to detection. One skilled in the art will recognize the detection methods where metabolite amplification is preferred.

The metabolite biomarkers of the present invention can be can be detected with standard imaging technology, for example, Magnetic Resonance Spectroscopy (MRS), a form of magnetic resonance imaging (MRI) based imaging. Proton magnetic resonance spectroscopy (1H-MRS) is one form of MRS. MRI imaging can be used for the detection and measurement of (e.g., concentration of) metabolite levels in tissues, such as the brain, or in fluids. Several of the metabolite biomarkers of the present invention, such as ATP, IMP, ADP and AMP, can be detected and measured using MRI analysis. The levels of the metabolite biomarkers of the present invention also can be detected with one or more of the following devices/methods for detecting metabolites: NMR, mass spectrometry (MS), gas chromatography (GC), High performance liquid chromatography (HPLC), capillary electrophoresis (CE), desorption electrospray ionization (DESI), laser ablation ESI (LAESI), ion-mobility spectrometry, electrochemical detection (coupled to HPLC), and Raman spectroscopy and radiolabel (when combined with thin-layer chromatography). Any assay that will detect the metabolite biomarkers of the present invention can be used. Further descriptions of detection methods are described above and also below in the Examples.

An advantage of the present diagnostic invention is that it is a rapid, relatively inexpensive and non-invasive method for diagnosing and assessing the prognosis of individuals to develop or be at risk for TBI, to have asymptomatic or early-stage TBI, or to be symptomatic of TBI. In some aspects, tests may be performed multiple times on the same subject to assess disease progress. One embodiment of the present inventive method comprises assaying a patient biological sample for a level of a specific metabolite(s) in the biological sample, wherein a different level of the specific metabolite(s) in the biological sample as compared to a statistically validated threshold for each specific metabolite(s) indicates TBI in the patient.

In certain aspects of the present invention, and as otherwise described herein, metabolite detection includes detecting the level of (e.g., the concentration of) one or more of the metabolites in the biological sample. The one or more metabolites in the biological sample may be at a different level than a statistically validated threshold for the one or more metabolites. The statistically validated threshold for the level of the specific metabolite(s) is based upon the level of each specific metabolite(s) in comparable control biological samples from a control population, e.g., from subjects that do not have TBI, or subjects that do not have head trauma. Various control populations are otherwise described herein. The statistically validated thresholds are related to the values used to characterize the level of the specific metabolite(s) in the biological sample obtained from the subject or patient. Thus, if the level of the metabolite is an absolute value, then the control value is also based upon an absolute value.

The statistically validated thresholds can take a variety of forms. For example, a statistically validated threshold can be a single cut-off value, such as a median or mean. Or, a statistically validated threshold can be divided equally (or unequally) into groups, such as low, medium, and high groups, the low group being individuals least likely to have traumatic brain injury and the high group being individuals most likely to have traumatic brain injury.

Statistically validated thresholds, e.g., mean levels, median levels, or “cut-off” levels, may be established by assaying a large sample of individuals in the select population and using a statistical model such as the predictive value method for selecting a positivity criterion or receiver operator characteristic curve that defines optimum specificity (highest true negative rate) and sensitivity (highest true positive rate). A “cutoff value” may be separately determined for the level of each specific metabolite assayed. Statistically validated thresholds also may be determined according to the methods described in the Examples hereinbelow.

The levels of the assayed metabolites in the patient biological sample may be compared to single control values or to ranges of control values. In one embodiment, a specific metabolite in a biological sample from a patient (e.g., a patient having or suspected of having TBI) is present at an elevated or reduced level (i.e., at a different level) than the specific metabolite in comparable control biological samples from subjects that do not have TBI when the level of the specific metabolite in the patient biological sample exceeds a threshold of one and one-half standard deviations above the mean of the concentration as compared to the comparable control biological samples. More preferably, a specific metabolite in a biological sample from a patient (e.g., a patient having or suspected of having TBI) is present at an elevated or reduced level (i.e., at a different level) than the specific metabolite in comparable control biological samples from subjects that do not have TBI when the level of the specific metabolite in the patient biological sample exceeds a threshold of two standard deviations above the mean of the concentration as compared to the comparable control biological samples. In another embodiment, a specific metabolite in a biological sample from a patient (e.g., a patient having or suspected of having TBI) is present at an elevated or reduced level (i.e., at a different level) than the specific metabolite in comparable control biological samples from subjects that do not have TBI when the level of the specific metabolite in the patient biological sample exceeds a threshold of three standard deviations above the mean of the concentration as compared to the comparable control biological samples.

If the level of a specific metabolite or metabolites in the patient biological sample are present at different levels than their respective statistically validated thresholds, then the patient is more likely to have TBI than are individuals with levels comparable to the statistically validated threshold. The extent of the difference between the subject's levels and statistically validated thresholds is also useful for characterizing the extent of the risk and thereby, determining which individuals would most greatly benefit from certain therapies, e.g., aggressive therapies. In those cases, where the statistically validated threshold ranges are divided into a plurality of groups, such as statistically validated threshold ranges for individuals at high risk of mild TBI, average risk of mild TBI, and low risk of mild TBI, the comparison involves determining into which group the subject's level of the relevant risk predictor falls.

A “reduced level” or an “elevated level” of a metabolite refer to the amount of expression or concentration of a metabolite in a biological sample from a patient compared to statistically validated thresholds, e.g., the amount of the metabolite in biological sample(s) from individual(s) that do not have TBI, have TBI (or a particular severity or stage of TBI), or have other reference diseases. For example, a metabolite has a “reduced level” in the serum from a subject when the metabolite is present at a lower concentration in the subject's serum sample than in serum from a subject who does not have TBI; and a metabolite has an “elevated level” in the serum from a subject when the metabolite is present at a higher concentration in the subject's serum sample than in serum from a subject who does not have TBI. For certain metabolites, elevated levels in a biological sample indicate the presence of or a risk for TBI; at the same time, other metabolites may be present in reduced levels in patients or subjects with TBI. In either of these example situations, metabolites are at a “different level” in TBI subjects versus healthy controls.

The differential expression of a particular biomarker indicating a diagnosis or prognosis for TBI may be more than, e.g., 1,000,000×, 100,000×, 10,000×, 1000×, 10×, 5×, 2×, 1× a particular statistically validated threshold, or less than, e.g., 0.5×, 0.1×, 0.01×, 0.001×, 0.0001×, 0.000001× a particular statistically validated threshold.

The metabolite biomarker methods of the present invention also can be combined with non-biomarker-based diagnostics to improve TBI diagnosis and for continued monitoring of the effect of treatment and/or the disease process. In one embodiment, a diagnosis of TBI using the present metabolite biomarker methods can be confirmed with or validated by structural information about the patient's brain. For example, the present methods can be performed either before or after brain imaging by one or more imaging modality, e.g., computerized tomography (CT) scanning, MRI, or PET imaging. Such imaging can be used to detect any subarachnoid or and intracerebral hemorrhaging, blood clots, subdural hematomas, edema (swelling), or necrosis of the brain tissue.

In another embodiment, the present metabolite biomarker methods can be combined with one or more other non-biomarker-based diagnostics of TBI, such as cognitive function testing of the patient, measurement of intracranial pressure or ICP (e.g., with an ICP monitor), measurement of an arteriovenous gradient, oxygen salivation measurements, measurement of lactate and pyruvate and then determining lactate/pyruvate ratios, or microdialysis catherization (microdialysis catherization allows for monitoring of energy related small molecules in body fluids). Also, the present metabolite biomarker methods can be combined with other biomarker-based diagnostics. More or less aggressive treatment can be administered to the patient depending on whether diagnosis using the present biomarker methods is confirmed by one or more of the alternative method of diagnosis.

Beyond disease prediction, the present metabolite biomarker methods also can include treating TBI in a subject. In addition to the detection and diagnostic methods described above, the inventive methods also can include, administering a therapeutically effective amount of a treatment for TBI to the diagnosed subject. That is, the present metabolite biomarker methods can be combined with the treatment of TBI, i.e., to indicate the initiation of one or more TBI therapies, discontinuation of one or more therapies, or an adjustment to one or more therapies (e.g., an increase or decrease to drug therapy, rehabilitation therapy, and the like.). The present metabolite biomarker methods also will allow for early prediction of TBI and for targeted therapy to reduce the severity or prevent altogether the development of major TBI complications, such as, seizure and brain damage. In response to the diagnosis of TBI, in some aspects of the method, a subject may be treated with one or more of TBI treatments (e.g., a drug, a surgery), or treated with a modification of an existing treatment, modified in response to the diagnosis or prognosis of TBI in that subject.

Another aspect of the invention is a method of providing medical services for a patient suspected of having or having TBI, including a physician, or other individual requesting a biological sample from and diagnostic information about the patient, wherein the diagnostic information is a level of one or more metabolites methionine sulfoxide, PC aa C 34:4, ATP, AMP, NAD+, ADP, IMP, spermidine, lysoPC a C20:3, C18:1, and proline in the biological sample; and the physician, or other individual administering a therapeutically effective amount of a treatment for TBI when the diagnostic information indicates that the level of the one or more metabolites in the biological sample is at a different level than a statistically validated threshold for the one or more metabolites.

There are several mechanisms of neuronal damage in TBI. As previously discussed, these include excessive release of excitatory neurotransmitters such as glutamine, which can lead to necrosis of the neuronal cells. Other mechanisms include the disturbance of energy metabolism leading to the interruption of the supply of oxygen and glucose to the neuronal cells to conduct critical cellular functions. Oxidative stress, a state of oxygen toxicity, causes impairment of cellular function through damage to important cellular macromolecules such as proteins, lipids and cell membranes in neurons and brain blood vessels. Oxidative stress is known to be one of the most important mechanisms of brain damage in TBI. These represent some of the mechanisms of ongoing damage post-trauma and they contribute to varying degrees. Variables that might determine which mechanisms predominate could include factors such as the type of trauma, e.g. closed versus penetrating injury, timing after injury and other intrinsic factors including genetic and overall health status. The metabolite markers of the present invention can be used to identify the predominant mechanism of injury and, thus, facilitate the selection of the appropriate therapy, e.g. the administration of progesterone for to treat inflammation in the TBI patient.

In some embodiments, the metabolite biomarker methods of the present invention can be combined with, and/or used for the selection of, various treatments for TBI. Different treatments for TBI can be ordered by a physician, or other healthcare provider, for a patient depending on the severity of the TBI (mild, moderate or severe) as indicated by the metabolite biomarker methods of the present invention. For example, if there is a statistically significant difference between only one or two of the present biomarkers and their controls, then the TBI might be considered to be mild, and a less aggressive or less invasive treatment ordered or administered by the physician, or other healthcare provider. Also, the amount and type of treatment can be selected depending on which and how many metabolic pathways/mechanisms (e.g., glutathione metabolism) are implicated by the results of using the present biomarker methods.

In some embodiments, the treatment is administered in a therapeutically effective amount. The therapeutically effective amount will vary depending upon a variety of factors including, but not limited to: the severity of the TBI (mild, moderate or severe) as indicated by the metabolite biomarker methods; the involved metabolic pathways/mechanisms as indicated by the metabolite biomarker methods; the age, body weight, general health, sex, and diet of the subject; the rate of excretion of any drug; any drug combination; and the mode and time of administration of the treatment.

One treatment for TBI is for a physician, healthcare provider, or other individual to advise (or order) the patient to reduce his/her exposure to further trauma to the brain. In an athletic context, an athlete might be “side-lined” for some period of time to prevent recurrence of an injury. In the instance where the present metabolite biomarker methods show mild TBI, the patient might be sided-lined for about 1-2 hours, about 2-5 hours, about 5-24 hours, or about 1-2 days. However, if the present metabolite biomarker methods indicate moderate or severe TBI, a patient might be side-lined for weeks or months, or permanently. Also, if the present metabolite biomarker methods indicate moderate or severe TBI, the physician, or other healthcare provider, could order a ventilator for the patient and/or the patient could be placed a ventilator (to ensure an adequate supply of oxygen), shunt installation could be ordered and/or a shunt could be installed in the patient, and/or surgery could be ordered or performed (e.g., to remove a portion of the patient's skull to allow for swelling). Another potential treatment that could be ordered or used to treat TBI is rehabilitation therapy.

The metabolite biomarker methods of the present invention also can be combined with, and/or used for the selection and administration of, various medications for the treatment of TBI. By using the present metabolite biomarker methods, a physician or other healthcare provide can determine whether medication is needed and, if so, the amount and type of the medication to be adminstered. Such medications could be used for the purpose of, for example, drawing fluid out of the brain and into blood vessels, decreasing the brain's metabolic requirements, and/or increasing blood flow to the injured tissues. Examples of TBI medications include, but are not limited to, medications for supportive therapy (pain killers, antibiotics, anti-seizure agents, etc.), progesterone, estrogen, erythropoiten (to increase red cell production and therefore oxygen carrying capacity of the blood), human chorionic gonadotropin (hCG), insulin growth factors, glyburide, glutamine antagonists, statins, immune suppressive agents (cyclosporins), anti-oxidants, prostocyclins, and anti-inflammatory agents. (Note, some of the medications currently are being evaluated in clinical trials). TBI medications treat different aspects of abnormalities that can result from TBI. The present biomarker metabolite methods can provide information on the development or predominance of particular abnormalities after TBI and, thus, can be used to guide choice of therapy and/or monitor therapeutic responses in individual cases. Further, if the levels of the present biomarkers indicate that the TBI is mild, treatment for the patient then might exclude toxic therapeutic agents.

Accurate prognostication is an important objective in TBI patient management. Accurate prognostication would be very beneficial in helping to assure appropriate patient and family counseling and to assess the likelihood of significant adverse outcomes including death and severe morbidities among survivors. In some aspects, the metabolite level is used to determine the efficacy of treatment received by a patient for TBI, that is, the metabolite levels of the patient may be assessed before treatment, and on one or more occasions after the administration of a treatment, to determine whether the treatment is effective. In particular, the present methods for diagnosing and treating also include performing the present metabolite biomarker methods on multiple occasions, i.e., to monitor the treatment effect and/or the brain condition of the patient over time. In particular, at one or more moments in time after initially performing the present metabolite biomarker methods, the present methods can again be performed and the results compared to results from an earlier-performed use of the present metabolite biomarker methods. A treatment for TBI can be administered before or after initially performing the present metabolite biomarker methods; and the course of treatment can be altered as indicated by the comparison(s). For example, if a TBI medication has been administered and, with the passage of time, there is a greater difference between the amount of a biomarker and its control, then a larger dose of the medicament (or surgery) might be indicated.

One embodiment of the present inventive method is the monitoring of treatment for TBI in a human patient, comprising: requesting a first biological sample from and first diagnostic information about the patient, wherein the first diagnostic information is a level of one or more metabolites methionine sulfoxide, PC aa C 34:4, ATP, AMP, NAD+, ADP, IMP, spermidine, lysoPC a C20:3, C18:1, and proline in the first biological sample; administering a therapeutically effective amount of a treatment for TBI to the patient; after administering the therapeutically effective amount of the treatment for TBI to the patient, requesting a second biological sample from and second diagnostic information about the patient, wherein the second diagnostic information is a level of one or more metabolites methionine sulfoxide, PC aa C 34:4, ATP, AMP, NAD+, ADP, IMP, spermidine, lysoPC a C20:3, C18:1, and proline in the second biological sample; and comparing the first diagnostic information and the second diagnostic information to determine whether the level of the one or more metabolites in the first biological sample is at a different level than the level of the one or more metabolites in the second biological sample.

Kits

Another embodiment of the present invention is a kit for diagnosing TBI. Kits that allow for the targeted measure of one or more metabolites would reduce both overall cost and turn-around time for a diagnosis of TBI.

In one embodiment, a biomarker panel is used to diagnose TBI. The panel would be configured to detect two or more of methionine sulfoxide, PC aa C 34:4, ATP, AMP, NAD+, ADP, IMP, spermidine, lysoPC a C20:3, C18:1, and proline. The inventive kit for diagnosing TBI may include (a) an MS-based or NMR-based array for detecting two or more of PC aa C 34:4, ATP, AMP, NAD+, ADP, IMP, spermidine, lysoPC a C20:3, C18:1, and proline; (b) a container for the biological sample; and (c) instructions for the method of detection.

In one embodiment, the present diagnostic methods and kits are useful for determining if and when medical treatments and therapeutic agents that are targeted at treating TBI should or should not be prescribed for an individual patient. Such medical treatments and therapeutic agents are discussed above and/or are known in the art, and will be ordered by or prescribed by a physician (or other healthcare provider) based on results of the inventive method and standard medical practices.

### EXAMPLES

Without further elaboration, it is believed that one skilled in the art can, using the preceding description, practice the present invention to its fullest extent. The following detailed examples describe how to perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Materials and Methods for Example 2

A standardized and validated closed head injury model of TBI in mice using a weight drop device was used for this study (Flierl M A, Stahel P F, Beauchanip K M, Morgan S J, Smith W R, Shohami E. Mouse closed head injury model induced by a weight-drop device. Nature Protocols 2009; 4; 1328-37). The study was limited to male mice to eliminate variability due to gender. The ages of the mice were between 10-12 weeks. Each mouse was kept in a separate cage for at least 7 days before subjected to study trauma. They were kept in a selective pathogen-free (SPF) environment in which the temperature and humidity were standardized (21° C. and 60% respectively. The length of light and dark cycles were kept at 12 hours each throughout the day and the animals were fed and watered ad libitum. National Institutes of Health (NIH) guidelines for the care of the animals are their use in experiments were adhered to. The study had been approved by the Institutional Animal Care and Use Committee of the University of Colorado.

A weight drop device of standard size was used for induction of localized head injury to the left brain hemisphere. Based on the published protocol. The animal was placed under isoflurane anesthesia induction, a longitudinal mid-line incision of the scalp was performed to expose the skull. The head was placed in a fixed position and standardized 333 g weight was dropped from a height of 2.5 cm onto the exposed skull to induce a blunt injury to the left hemisphere. Supportive care was provided including oxygenation with 100% 02 until the mouse was awake. Intraperitoneal fentanyl injection of 0.05 mg/kg was given just prior to the weight drop and then in a dose of 0.01 mg/kg every 12 hours. For the sham-operated controls the entire procedure surgery, oxygen support, anesthesia, and analgesia with the exception of the head-trauma step. The complete details above were as described in a prior publication (Neher M D, Rich M C, Keene C N, Weckbach S, Bolden A L, Losacco J T et al. Deficiency of complement receptors CR2/CR1 in Cr2-1-mice reduces the extent of secondary brain damage after closed head injury. J. Neuroinflammation. 2014; 11:1-13).

Animals were sacrificed at 4 and 24 hours after surgery. Decapitation was performed under isoflurane anesthesia. Blood was obtained by cardiac puncture just prior to decapitation. Serum was obtained by centrifugation at 10,000 g for 10 minutes at 4° C. and the serum was separated and stored at −80° C. The hemispheres were separated and the right hemisphere which had not been subjected to trauma, and cerebellum were removed and stored.

Brain Tissue Extraction

Whole mouse brains (collected in separate 5 ml sterile Eppendorf tubes to avoid cross contamination) were homogenized in 10 ml/g (wet weight) of ice cold 50% methanol using a Caframo homogenizer (Ontario, Canada) for approximately 1 min until a smooth homogenate was formed, without heat generation. The homogenizer probe was washed before and after each sample. The samples were subsequently mixed for 30 seconds and sonicated for 20 min at 4° C., and the protein removed by centrifugation at 4000 g at 4° C. for 20 min. The supernatant was evaporated to dryness using a Savant DNA Speedvac (Thermo Scientific, USA) and reconstituted in 570 μl of 50 mM sodium phosphate buffer, 70 μl of D2O and 60 μl of D2O containing 0.01 M 4,4-Dimethyl-4-silapentane-1-sulfonic acid (DSS). The samples were mixed for 30 seconds and centrifuged for 15 min at 4° C. for 20 min to remove any debris. 650 μl of supernatant was transferred to a 5 mm NMR tube for analysis.

Nuclear Magnetic Resonance (NMR) Spectroscopy Analysis (by 1H NMR)

All 1H NMR experiments were recorded at 298.0 (±0.05) K on a 900 MHz Bruker Avance spectrometer (Bruker-Biospin, USA) equipped with a cryo-probe operating at 900.13 MHz. In a randomized order, all spectra were acquired manually using a previously reported pulse sequence (Mercier, P., Lewis, M. J., Chang, D., Baker, D. and Wishart, D. S. (2011) Towards automatic metabolomic profiling of high-resolution one-dimensional proton NMR spectra J. Biomol NMR 49:307-32). One hundred and twenty eight transients were acquired for each spectrum. Chemical shifts (d) are reported in parts per million (ppm) of the operating frequency. The singlet produced by the DSS methyl groups was used as an internal standard for chemical shift referencing (set to 0 ppm, concentration 500 uM) and for quantification all 1H-NMR spectra were processed and analyzed using the Chenomx NMR Suite Professional Software package version 7.7 (Chenomx Inc, Edmonton, AB). The Chenomx NMR Suite allowed for qualitative and quantitative analysis of the NMR spectra by manually fitting spectral signatures from an internal database to the spectrum. Specifically, the metabolite spectral fitting was done using the standard Chenomx 800 MHz metabolite library, which was the closest to the collection field strength of 900 MHz. Spectra were assigned simultaneously to maximize assignment uniformity. Typically, more than 90% of the spectral area was accounted for and fit to metabolites using the Chenomx spectral analysis software.

Pathway Topology and Heatmap Analysis

Metabolites that were found to be significantly different (p<0.05) between sham and TBI mice were analyzed using the pathway topology search tool in Metaboanalyst (v 2.0) (Xia, J., Psychogios, N., Young, N. and Wishart, D. S. (2009) MetaboAnalyst: a web server for metabolomic data analysis and interpretation. Nucl. Acids Res. 37, W652-660; Xia, J., Mandal, R., Sinelnikov, I., Broadhurst, D., and Wishart, D. S. (2012) MetaboAnalyst 2.0—a comprehensive server for metabolomic data analysis. Nucl. Acids Res. 40, W127-W133; Xia, J., Sinelnikov, I., Han, B., and Wishart, D. S. (2015) MetaboAnalyst 3.0—making metabolomics more meaningful. Nucl. Acids Res. (DOI: 10.1093/nar/gkv380)). The library pathway chosen was Mus musculus (mouse) and all compounds in selected pathways were used when referencing the specific metabolome. Fisher's exact test was applied to the selected algorithm for over-representation analysis and relative betweeness centrality was chosen for the pathway topology testing. Pathways that had a Holm corrected p-value and false discovery rate all <0.05 were considered to be altered due to the TBI. Heatmaps were produced using the statistical analysis tool in Metaboanalyst (v 2.0). The data were normalized to the median and range scaled. Subsequently for the Hetamap analyses the distance measured was Euclidean and the cluster algorithm used was Ward's.

Statistical Analysis

Two separate groups of comparisons were performed, between the TBI group sacrificed at 4 hours (early) after trauma versus those sacrificed at 24 hours (late). The second comparison was between TBI versus sham-operated controls.

Log transformation and Pareto scaling were performed to normalize the data. Mean and SD metabolite concentrations were compared between different groups including TBI versus normal and between the two TBI groups. Metabolite concentrations were expressed as μM/L. Principal components analysis (PCA) is a dimensional reduction technique whereby a large number of potential predictor variables (metabolites) is reduced to the few that best distinguishes the study from the control groups. The separation achieved between the two groups is displayed on a 2-D and 3-D cluster plots. On the 2-D plot, the two principal components or metabolites are displayed on the x- and y-axes. The x-axis contains the values for the most discriminating metabolite (‘principal component’) and the y-axis contains the second most discriminating metabolite. For the 3-D plot the third most predictive metabolite is in addition displayed on the z-axis. (Wishart D S. Computational approaches to metabolomics methods Mol Biol 2010; 593:283-313.). Clustering and separation of data points of the two groups indicate the capacity of the principal components used on the graph to distinguish case from control groups. The greater the visual separation of the two clusters or groups of cases the greater the diagnostic accuracy of the principal components. Partial least squares discriminant analysis (PLS-DA) is an additional technique for maximizing the separation between the groups by rotating the principal components used to identify the optimal combination of metabolites to achieve maximum separation of the groups (Wishart D S. Computational approaches to metabolomics methods Mol Biol 2010; 593:283-313).

The inventors subsequently performed permutation testing (2000 rounds of testing) to establish whether the observed discrimination between the groups was statistically significant. PCA, PLS-DA and permutation testing were done with the MetaboAnalyst computerized program (Xia J, Psychogiss N, Young N, Wishart D S. Metabo Analyst: a web server for metabolomic data analysis and interpretation. Nucleic acids Res 2009; 37:W652-W660). A variable importance ranking in projection (VIP) score plot is a graphic ranking of metabolites based on their relative importance. The higher the VIP score on the x-axis, the more important the metabolite. Only metabolites with VIP≧1.0 are listed. (Wishart D S. Computational approaches to metabolomics methods Mol Biol 2010; 593: 283-313). P-values were calculated based on the t-test. For non-normal distributions the p-values were based on Mann-Whitney U test. P values <0.05 were considered statistically significant. Bonferroni corrected p-values (p=0.05/number of metabolites) was used to correct for multiple comparisons.

Logistic regression analysis was used to determine the optimal combination of metabolites needed to distinguish the different study groups. Predictor variables considered in the regression were limited to those with p<0.3 on univariate comparisons. Variable selection methods used Least Absolute Shrinkage and Selection Operator (LASSO) (Tibshirani R. Regression shrinkage and selection vice Lasso. J. R. Statist SOC 1996; 58: 267-88) and stepwise variable selection in the logistic regression process to optimize model components (Hastie T, Tibshirani R, Friedman J. The Elements of Statistical Learning: Data Mining, Inference, and Prediction. Second Edition Springer Series in Statistics. Springer-Verlaf, 2009. Springer, NY). Ten-fold cross-validation (CV) optimization was used for deciding on the final predictor variables to be included in the model. Inclusion in the model required that a particular predictor was selected >8 times out of the 10 CV exercises.

The area under the receiver operating characteristics curve (AUROC or AUC) was calculated using previously described techniques (Xia J, Broadhurst D I, Wilson M, Wishart D S. Tranlational biomarker discovery in clinical metabolomics: an introductory tutorial. Metabolomics 2013; 9:280-99). Sensitivity and specificity values were also calculated.

### Example 2

### Results for Brain Tissue Samples

There were a total of 18 mice of which 10 had TBI and 8 were sham-operated. Table 1 (below) compares the TBI versus control groups. There was no significant difference in intervals between groups or in body weight. The early TBI mice (4 hours between treatment and sacrifice) had higher body weights when compared to the late group (sacrificed after 24 hours). Weight mean (SD) were 26.5(1.25) g versus 23.8(1.46) g, p=0.013 respectively.

TABLE 1
Comparison of TBI and Sham-operated Controls

| Parameter | TBI | Sham (Control) | p value | Method |
|---|---|---|---|---|
| Number of cases | 10 | 8 | | — |
| Interval Time, n (%) | | | | |
| 4 Hr | 5 (50) | 4 (50) | 1.000 | Fisher's |
| 24 Hr | 5 (50) | 4 (50) | | Exact |
| Weight | 25.1 (1.93) | 24.7 (1.90) | 0.624 | t-test |

There were a total of 46 metabolites measured using NMR. Table 2 (below) compares the mean (SD) metabolite concentrations measured in μM/L between TBI cases and sham treated controls. A total of 29 metabolites were found in significantly different concentrations in the contralateral lobe of the TBI group compared to controls with a Bonferroni adjusted P-value of <0.05. A total of 15 metabolites were elevated in the brain of TBI cases and 14 were significantly reduced. Given the small number of cases in each group, a similar comparison between the late- and early-TBI groups did not yield any statistically significant differences.

TABLE 2
NMR Analysis with Univariate Metabolite comparisons: TBI versus Sham-Operated mice.

| | TBI vs Sham | | | TBI | Fold | q-value |
|---|---|---|---|---|---|---|
| Metabolite | TBI | Sham (Control) | p-value | /Sham | Change | (FDR) |
| Number of cases | 10 | 8 | — | — | — | — |
| 4-Aminobutyrate | 2021.51 (60.97) | 2913.37 (262.69) | <0.001 | Down | −1.44 | 0 |
| ADP | 126.24 (76.07) | 23.57 (9.17) | <0.001 | Up | 5.36 | 0 |
| AMP | 222.96 (55.42) | 12.97 (15.74) | <0.001 | Up | 17.19 | 0.001 |
| ATP | 83.88 (15.89) | 48.79 (31.74) | 0.009 | Up | 1.72 | 0.015 |
| Acetate | 2190.92 (395.92) | 3076.59 (561.18) | 0.002 | Down | −1.4 | 0.004 |
| Adenine | 50.53 (18.80) | 38.60 (25.00) | 0.328 | Up | 1.31 | 0.347 |
| Alanine | 709.51 (52.58) | 852.31 (72.90) | <0.001 | Down | −1.2 | 0 |
| Anserine | 123.99 (32.98) | 115.22 (61.35) | 1 | Up | 1.08 | 0.734 |
| Ascorbate | 1185.69 (124.15) | 928.82 (85.31) | <0.001 | Up | 1.28 | 0 |
| Aspartate | 3239.54 (357.03) | 4276.15 (440.87) | <0.001 | Down | −1.32 | 0 |
| Caffeine | 107.49 (28.20) | 109.77 (13.05) | 0.762 | Down | −1.02 | 0.854 |
| Choline | 151.44 (19.08) | 313.68 (26.22) | <0.001 | Down | −2.07 | 0 |
| Creatine | 5594.17 (315.71) | 5056.00 (358.62) | 0.003 | Up | 1.11 | 0.006 |
| Ethanolamine | 140.57 (15.35) | 155.80 (42.28) | 0.091 | Down | −1.11 | 0.435 |
| Fucose | 92.72 (10.86) | 80.41 (32.40) | 0.477 | Up | 1.15 | 0.413 |
| GTP | 31.15 (14.31) | 23.05 (9.34) | 0.315 | Up | 1.35 | 0.26 |
| Glutamate | 6491.68 (579.13) | 5599.00 (474.71) | 0.002 | Up | 1.16 | 0.004 |
| Glutamine | 2637.10 (263.56) | 2525.32 (265.42) | 0.315 | Up | 1.04 | 0.444 |
| Glutathione | 701.80 (62.25) | 297.92 (99.24) | <0.001 | Up | 2.36 | 0 |
| Glycine | 930.09 (322.70) | 941.77 (428.51) | 0.374 | Down | −1.01 | 0.948 |
| Hypoxanthine | 323.45 (57.11) | 766.34 (118.05) | <0.001 | Down | −2.37 | 0 |
| IMP | 66.09 (13.13) | 16.69 (8.47) | <0.001 | Up | 3.96 | 0 |
| Inosine | 360.89 (62.65) | 653.42 (176.47) | <0.001 | Down | −1.81 | 0 |
| Isobutyrate | 8.54 (1.67) | 7.52 (1.61) | 0.722 | Up | 1.13 | 0.287 |
| Isoleucine | 41.02 (6.64) | 78.98 (11.30) | <0.001 | Down | −1.93 | 0 |
| Lactate | 8476.44 (729.61) | 8370.93 (276.50) | 1 | Up | 1.01 | 0.729 |
| Leucine | 77.37 (6.07) | 197.12 (55.10) | <0.001 | Down | −2.55 | 0 |
| Methanol | 152.48 (16.78) | 117.69 (16.38) | 0.001 | Up | 1.3 | 0.002 |
| N-Acetylaspartate | 2221.28 (216.59) | 808.29 (517.53) | <0.001 | Up | 2.75 | 0 |
| NAD+ | 33.70 (12.95) | 1.43 (0.45) | <0.001 | Up | 23.57 | 0.001 |
| Niacinamide | 175.76 (11.48) | 247.01 (18.05) | <0.001 | Down | −1.41 | 0 |
| O-Acetylcarnitine | 19.68 (4.66) | 10.97 (5.61) | 0.002 | Up | 1.79 | 0.004 |
| O-Phosphocholine | 397.70 (49.57) | 465.47 (39.02) | 0.012 | Down | −1.17 | 0.019 |
| Pantothenate | 38.97 (9.02) | 41.02 (9.20) | 0.306 | Down | −1.05 | 0.703 |
| Phenylalanine | 62.88 (35.37) | 92.93 (13.90) | 0.016 | Down | −1.48 | 0.025 |
| Propylene glycol | 90.57 (11.29) | 116.40 (61.37) | 0.505 | Down | −1.29 | 0.353 |
| Succinate | 500.69 (41.52) | 279.67 (55.47) | <0.001 | Up | 1.79 | 0 |
| Taurine | 6196.13 (325.13) | 5836.26 (359.35) | 0.05 | Up | 1.06 | 0.062 |
| Tyrosine | 54.89 (16.91) | 104.23 (14.37) | <0.001 | Down | −1.9 | 0 |
| UDP-galactose | 49.65 (17.18) | 56.05 (20.06) | 0.696 | Down | −1.13 | 0.535 |
| UDP-glucose | 41.45 (9.08) | 25.98 (13.50) | 0.009 | Up | 1.6 | 0.015 |
| Uracil | 122.44 (16.14) | 138.26 (17.80) | 0.146 | Down | −1.13 | 0.098 |
| Urea | 876.64 (115.86) | 965.09 (246.83) | 0.408 | Down | −1.1 | 0.44 |
| Valine | 69.06 (5.39) | 105.88 (19.35) | <0.001 | Down | −1.53 | 0 |
| myo-Inositol | 3557.29 (233.15) | 3356.24 (296.69) | 0.083 | Up | 1.06 | 0.182 |
| sn-Glycero-3- | 696.41 (63.80) | 416.84 (45.91) | <0.001 | Up | 1.67 | 0 |
| phosphocholine | | | | | | |

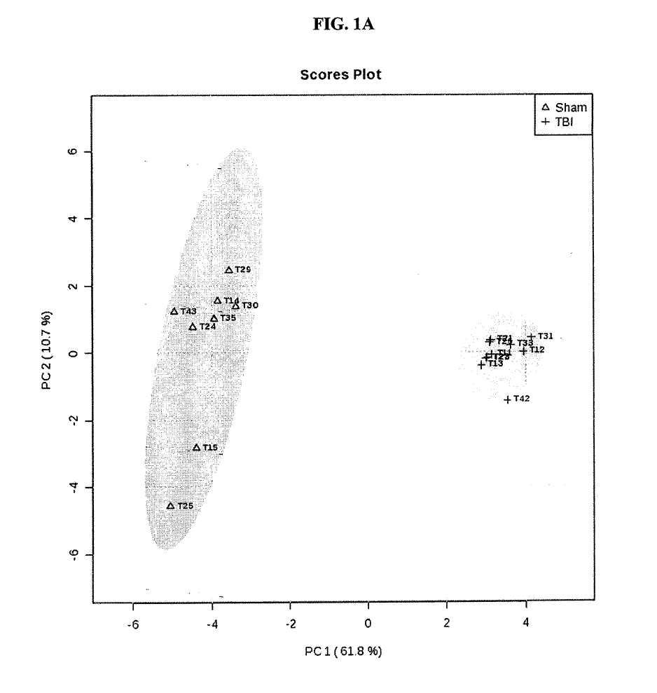

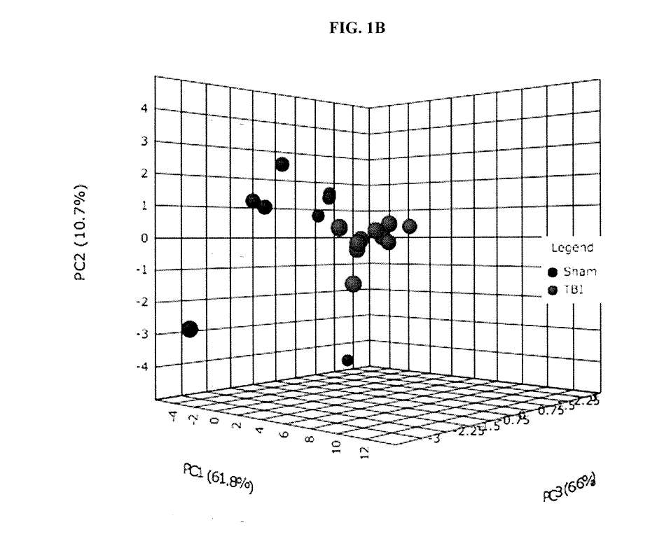

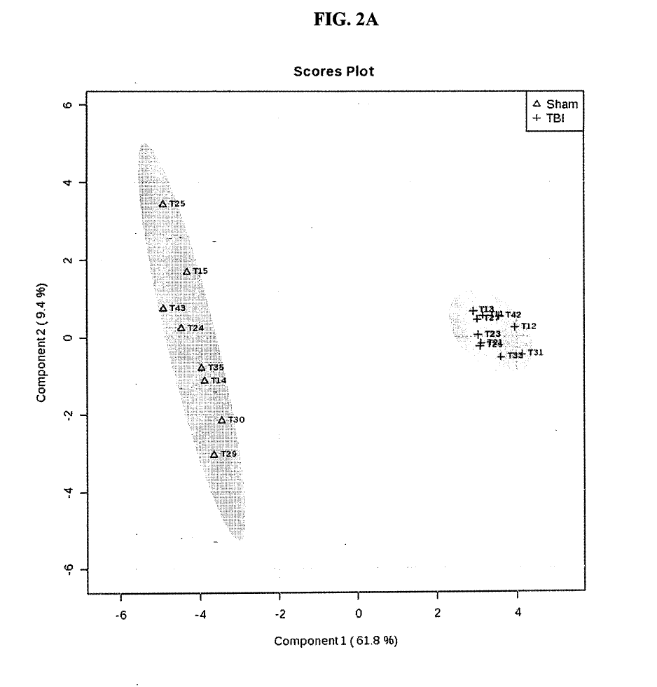

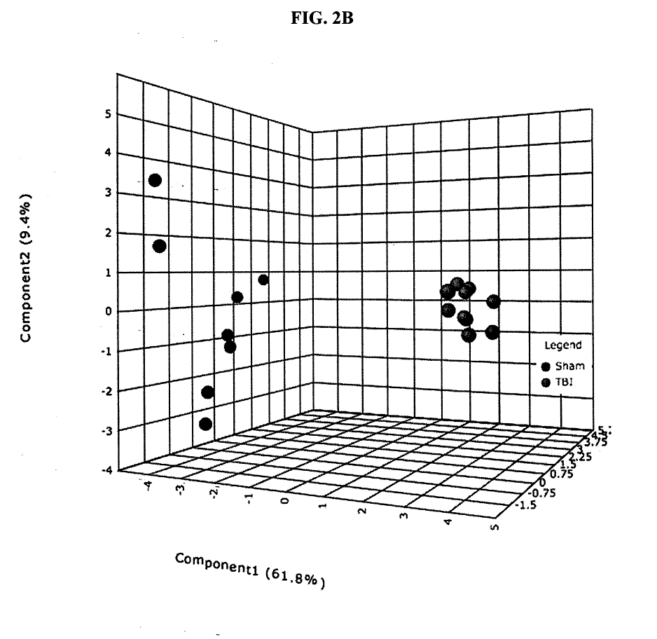

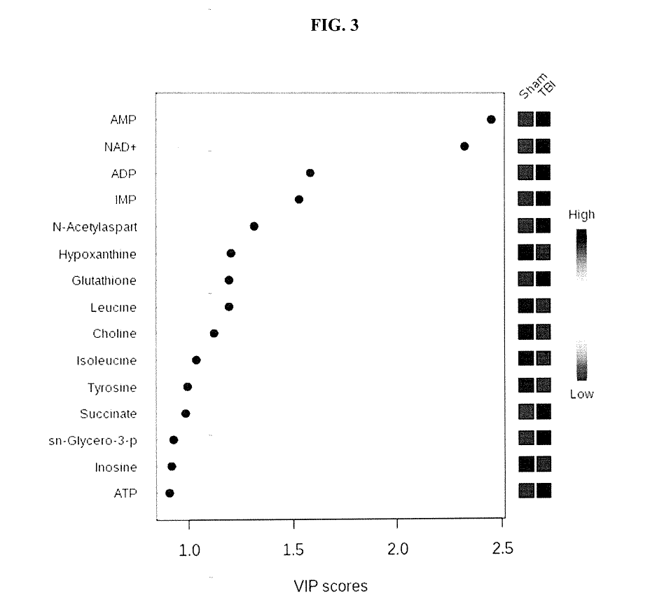

FIGS. 1A and 1B show the 2-D and 3-D PCA plot for the TBI versus sham-operated group. There is clear separation of the two groups. Even clearer separation was achieved on the PLS-DA plots (see, FIGS. 2A and 2B). Permutation testing (2000 repeats) for the PLS-DA model had a p-value=0.024, indicating that the observed separation was indeed statistically significant and that it was not due to overfitting. The VIP plot is shown in FIG. 3. As shown in this figure, AMP was the most significant discriminator of TBI and was elevated in affected cases, followed by NAD+, ADP and IMP. All three metabolites were elevated in TBI cases.

Similar analyses were performed for the early-TBI versus late-TBI groups. Each group contained only 5 cases however. Some separation was observed on the PCA and also the PLS-DA plots for this comparison (not shown). Permutation testing (2000 repeats) was performed to determine whatever the metabolites were able to distinguish the early- from late-TBI groups. The P-value (0.0899) however was not statistically significant. This is very likely due to the small number of samples available in each category.

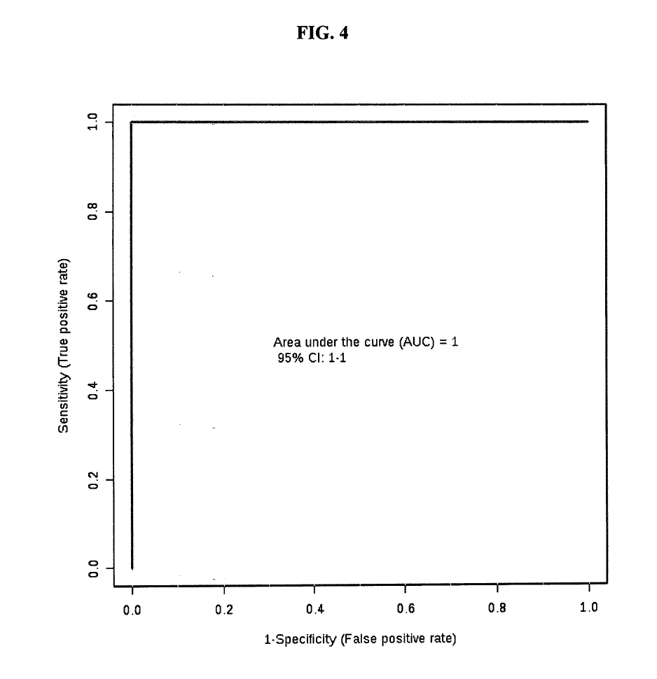

Due to the complete separation between cases and controls, ROC curve analysis was performed with the PLS-DA model instead of the logistic regression model (Metabo Analyst Program) (Xia J, Psychogiss N, Young N, Wishart D S. Metabo Analyst: a web server for metabolomic data analysis and interpretation. Nucleic Acids Res 2009; 37:W652-W660). FIG. 4 shows the ROC plot for distinguishing TBI as a group from sham-operated mice. Perfect separation of the group was achieved with the combination of ADP, AMP, NAD+, IMP as biomarkers. The AUC (95% CI) was 1.0 (1.0,1.0). The inventors further performed permutation analysis in order to confirm that the ROC analysis based on the PLS-DA model was significant. One thousand repeats of permutation testing was performed and the p-value=0.03.

To evaluate the ability of metabolites to distinguish early- from late-TBI cases. Based on the PLS-DA model (not shown), four metabolites: tyrosine, acetate, hypoxanthine, and ADP were used for prediction. The AUC (95% CI)=0.952 (0.75,1.0). Stringent testing using permutation analysis (1000 repeats) were performed for the ROC analysis. Despite the high AUC in the initial analysis the p-value=0.40 was not significant on permutation analysis. Again this is most likely a result of insufficient study power and the need for larger numbers of cases for this particular comparison.

Initial pathway analysis yielded a total of 22 biochemical pathways for which there was significant alterations in the combined TBI group compared to controls, with FDR P-value of <0.05. Using the more stringent criteria outlined in the materials and methods section (Holm adjusted p-value <0.001, fdr p-value <0.001 and impact >0.1), this list was reduced to 7-pathway (see, Table 3 below). Disturbance of purine metabolism appeared to be the most significantly affected in TBI brains.

TABLE 3
Pathway analysis: Brain Metabolic Pathways significantly disturbed with TBI.

| | Total | | | Holm | | |
|---|---|---|---|---|---|---|
| Pathway | Compounds | Hits | Raw p | adjusted | FDR | Impact |
| Purine metabolism | 68 | 9 | 3.89E−13 | 1.52E−11 | 1.52E−11 | 0.23 |
| Alanine, aspartate | 24 | 5 | 1.23E−09 | 4.04E−08 | 6.83E−09 | 0.46 |
| and glutamate | | | | | | |
| metabolism | | | | | | |
| Glutathione | 26 | 2 | 1.86E−09 | 5.94E−08 | 9.05E−09 | 0.37 |
| metabolism | | | | | | |
| Valine, leucine and | 11 | 2 | 6.66E−09 | 2.06E−07 | 2.6E−08 | 0.67 |
| isoleucine | | | | | | |
| biosynthesis | | | | | | |
| Glycine, serine and | 31 | 3 | 2.24E−07 | 6.50E−06 | 7.95E−07 | 0.27 |
| threonine metabolism | | | | | | |
| Nicotinate and | 13 | 2 | 3.2E−06 | 8.97E−06 | 1.04E−05 | 0.45 |
| nicotinamide | | | | | | |
| metabolism | | | | | | |
| Tyrosine metabolism | 44 | 1 | 1.58E−04 | 4.10E−03 | 4.10E−04 | 0.14 |

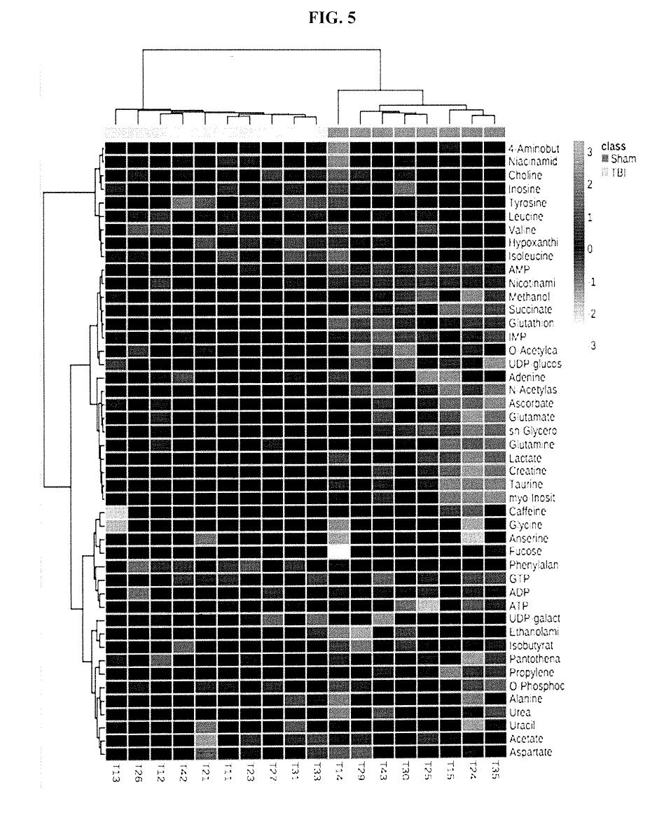

The heat map (FIG. 5) visually demonstrates the concentration differences between TBI overall and sham controls. In addition, the direction of change of these metabolites in TBI is indicated on the map.

### Example 3

### Materials and Methods for Example 4

Serum from the mice described in Examples 1 and 2 was analyzed. Example 4 reports serum data from the same mice using Liquid Chromatography and Mass Spectrometry (LC-MS/MS) of serum. The analysis of brain tissue used a different metabolomics platform-namely Nuclear Magnetic Resonance (NMR). Examples 2 and 4 identify some overlapping and some different metabolites.

The technique of inducing TBI and the description of the preparation, monitoring and euthanizing of the animals is described in Example 1. Blood was obtained by cardiac puncture just prior to decapitation. Serum was obtained by centrifugation at 10,000 g for 10 minutes at 4° C. and the serum was separated and stored at −80° C.

Metabolomic Analysis: Combined Direct Injection (DI) and Liquid Chromatography-Mass Spectrometry (LC-MS/MS) Compound Identification and Quantification

Targeted quantitative metabolomics analysis of the serum was performed by combining direct injection mass spectrometry (AbsoluteIDQ™ Kit) with a reverse-phase LC-MS/MS Kit. The Kit is a commercially available from BIOCRATES Life Sciences AG (Austria). In combination with an ABI 4000 Q-Trap (Applied Biosystems/MDS Sciex) mass spectrometer targeted identification and quantification of up to 180 different endogenous metabolites including amino acids, acylcarnitines, biogenic amines, glycerophospholipids, sphingolipids and sugars. Derivatization and extraction of analytes, and the selective mass-spectrometric detection using multiple reaction monitoring (MRM) pairs was performed. Isotope-labeled internal standards and other internal standards are integrated in Kit plate filter for metabolite quantification. The AbsoluteIDQ kit contains a 96 deep-well plate with a filter plate attached with sealing tape, and reagents and solvents used to prepare the plate assay. Of the first 14 wells in the Kit were used as follows: one for the blank, three zero samples, seven standards and three quality control samples provided with each Kit.

All of the serum samples were analyzed with the AbsoluteIDQ kit as described in the AbsoluteIDQ user manual. Serum samples were thawed on ice and were vortexed and centrifuged at 13,000×g. Ten μL of each serum sample was loaded onto the center of the filter on the upper 96-well kit plate and dried in a nitrogen stream. Subsequently, 20 μL of a 5% solution of phenyl-isothiocyanate was added for derivatization. After incubation, the filter spots were dried again using an evaporator. Extraction of the metabolites was then achieved by adding 300 μL methanol containing 5 mM ammonium acetate. The extracts were obtained by centrifugation into the lower 96-deep well plate, followed by a dilution step with kit MS running solvent.

Mass spectrometric analysis was performed on an API4000 Qtrap® tandem mass spectrometry instrument (Applied Biosystems/MDS Analytical Technologies, Foster City, Calif.) equipped with a solvent delivery system. The samples were delivered to the mass spectrometer by a LC method followed by a direct injection (DI) method. The Biocrates MetIQ software was used to control the entire assay workflow, from sample registration to automated calculation of metabolite concentrations to the export of data into other data analysis programs. A targeted profiling scheme was used to quantitatively screen for known small molecule metabolites using multiple reaction monitoring, neutral loss and precursor ion scans.

Pathway Topology and Heatmap Analysis

Metabolites that were found to be significantly different (p<0.05) between sham and TBI mice were analyzed using the pathway topology search tool in Metaboanalyst (v 2.0) (Xia, J., Psychogios, N., Young, N. and Wishart, D. S. (2009) MetaboAnalyst: a web server for metabolomic data analysis and interpretation. Nucl. Acids Res. 37, W652-660; Xia, J., Mandal, R., Sinelnikov, I., Broadhurst, D., and Wishart, D. S. (2012) MetaboAnalyst 2.0—a comprehensive server for metabolomic data analysis. Nucl. Acids Res. 40, W127-W133; Xia, J., Sinelnikov, I., Han, B., and Wishart, D. S. (2015) MetaboAnalyst 3.0—making metabolomics more meaningful. Nucl. Acids Res. (DOI: 10.1093/nar/gkv380)). The library pathway chosen was Mus musculus (mouse) and all compounds in selected pathways were used when referencing the specific metabolome. Fisher's exact test was applied to the selected algorithm for over-representation analysis and relative betweeness centrality was chosen for the pathway topology testing. Pathways that had a Holm corrected p-value and false discovery rate all <0.05 were considered to be altered due to the TBI. Heatmaps were produced using the statistical analysis tool in Metaboanalyst (v 2.0). The data were normalized to the median and range scaled. Subsequently for the Heatmap analyses the distance measured was Euclidean and the cluster algorithm used was Ward's.

Statistical Analysis

The TBI group sacrificed at 4 h (early) after trauma and those sacrificed at 24 h (late) were compared. The overall TBI (early+late) group was also compared to sham-operated controls.

Log transformation and Pareto scaling were used to normalize the data. Mean and SD of metabolite concentrations were compared between the groups. Principal component analysis (PCA), Partial least squares discriminant analysis (PLS-DA), a variable importance ranking in projection analysis (VIP) were per-formed using a subset of metabolites that had a p value <0.3 based on univariate comparisons. For the generated PLS-DA model, permutation testing was performed to determine the statistical likelihood that the observed separation is either due to chance or real differences between cases and controls. This was accomplished by randomly relabeling the data and by rerunning the PLS-DA analysis. Permutation testing with 2000 repetitions were performed to establish whether the observed discrimination between the groups was statistically significant (Wishart 2010; Xia et al. 2009). Only the top metabolites (n=15), as displayed in the VIP plot were listed. For the comparison of each metabolite, the p values were calculated using a Mann-Whitney U test. p value <0.05 was considered statistically significant. Bonferroni corrected p value=0.0003 was used for multiple testing correction. In addition the q-value or FDR was used to determine significance (p<0.05).

Logistic regression analysis was used to determine the optimal metabolite combination for TBI prediction. Predictor variables used were limited to those with p value <0.3 based on univariate comparisons. Variable selection methods using Least Absolute Shrinkage and Selection Operator (LASSO) (Tibshirani 1996) was used and stepwise variable selection in the logistic regression calculation was employed to optimize the model components (Hastie et al. 2009). A k-fold cross-validation (CV) technique was used to ensure that the logistic regression models were robust (Xia et al. 2013). In k-fold CV, the entire sample is randomly divided into k equal sized subsets. Of the k subsets, only one subset is used as the validation data for testing the model, and the remaining (k−1) subsets are used as training set to generate the model. The end result is a robust, optimal and parsimonious biomarker model. The predictive ability of the model is then tested independently on the validation group which was not used in model generation. The performance of the model over these rounds is then averaged. The five-fold cross-validation (CV) with LASSO was used to determine the final set of coefficients and variables to be included in the model. Inclusion in the model required that a given predictor be selected >4 times out of the 5 CV exercises. The area under the receiver operating characteristics curve (AUROC or AUC) and sensitivity and specificity values were also calculated (Xia et al. 2013). In the case of TBI prediction, it may be better to over predict. This translates to maximizing sensitivity providing the best results. So, both the threshold (or cutoff) values of the generated logistic regression model for maximized sensitivity and the best sum of sensitivity and specificity were considered.

### Example 4

### Results for Serum Samples

The LC-MS/MS initially yielded 158 metabolites. After excluding metabolites in which values were missing in >20% of samples, the final number fell to 150 metabolites. Serum samples were available from a total of ten TBI mice and eight sham-operated control mice. Of these, five TBI and four controls had blood sampling done at 4 h post TBI while five cases and four controls were sampled at 24 h. Thirty-six of a total of 150 (24.0%) metabolites had significant concentration differences in TBI compared to control mice (Table 4).

TABLE 4
LC-MS/MS Analysis with Univariate Metabolite comparisons:
TBI versus Sham-Operated mice (Serum Data).

| | TBI vs Sham | | p- | q-value | Fold | TBI/ |
|---|---|---|---|---|---|---|
| Metabolite | TBI (n = 10) | Sham (n = 8) | value | (FDR) | Change | Sham |
| C0 | 24.02 (5.85) | 30.95 (2.48) | 0.0160 | 0.0539 | −1.29 | ↓ |
| C14 | 0.09 (0.02) | 0.07 (0.01) | 0.0430 | 0.1670 | 1.23 | ↑ |
| C14:1 | 0.10 (0.01) | 0.09 (0.01) | 0.0060 | 0.0472 | 1.20 | ↑ |
| C14:2 | 0.03 (0.01) | 0.02 (0.00) | 0.0090 | 0.0712 | 1.30 | ↑ |
| C16 | 0.23 (0.05) | 0.17 (0.02) | 0.0210 | 0.0525 | 1.38 | ↑ |
| C16:1 | 0.06 (0.01) | 0.05 (0.01) | 0.0160 | 0.0561 | 1.36 | ↑ |
| C18 | 0.06 (0.01) | 0.05 (0.00) | 0.0040 | 0.0525 | 1.28 | ↑ |
| C18:1 | 0.21 (0.06) | 0.14 (0.02) | 0.0020 | 0.0515 | 1.56 | ↑ |
| C18:2 | 0.10 (0.02) | 0.07 (0.01) | 0.0010 | 0.0525 | 1.37 | ↑ |
| C2 | 25.65 (4.90) | 19.81 (1.77) | 0.0010 | 0.0472 | 1.29 | ↑ |
| C3 | 0.84 (0.34) | 0.75 (0.21) | 0.9650 | 0.7212 | 1.12 | ↑ |
| C5—OH (C3-DC-M) | 0.08 (0.01) | 0.07 (0.01) | 0.3600 | 0.6136 | 1.04 | ↑ |
| C4 | 2.18 (0.64) | 2.17 (0.42) | 0.7620 | 0.9943 | 1.00 | ↑ |
| C3-DC (C4—OH) | 0.16 (0.04) | 0.11 (0.01) | 0.0010 | 0.0436 | 1.39 | ↑ |
| C5 | 0.24 (0.07) | 0.25 (0.06) | 0.7620 | 0.9321 | −1.03 | ↓ |
| C6 (C4:1-DC) | 0.23 (0.08) | 0.24 (0.08) | 0.8290 | 0.9321 | −1.03 | ↓ |
| lysoPC a C16:0 | 307.32 (44.30) | 331.92 (70.74) | 0.4600 | 0.6124 | −1.08 | ↓ |
| lysoPC a C16:1 | 7.22 (2.63) | 8.30 (1.16) | 0.1460 | 0.5365 | −1.15 | ↓ |
| lysoPC a C17:0 | 3.73 (0.68) | 4.40 (1.37) | 0.4080 | 0.6249 | −1.18 | ↓ |
| lysoPC a C18:0 | 125.12 (23.83) | 128.94 (32.89) | 0.9650 | 0.9283 | −1.03 | ↓ |
| lysoPC a C18:1 | 52.36 (14.19) | 59.90 (6.96) | 0.1010 | 0.4721 | −1.14 | ↓ |
| lysoPC a C18:2 | 140.24 (38.83) | 198.88 (35.71) | 0.0090 | 0.0525 | −1.42 | ↓ |
| lysoPC a C20:3 | 6.47 (2.18) | 9.94 (1.49) | 0.0010 | 0.0472 | −1.54 | ↓ |
| lysoPC a C20:4 | 35.63 (6.56) | 46.77 (3.71) | 0.0010 | 0.0436 | −1.31 | ↓ |
| lysoPC a C24:0 | 0.76 (0.12) | 0.82 (0.11) | 0.4600 | 0.6124 | −1.07 | ↓ |
| lysoPC a C26:0 | 0.56 (0.14) | 0.66 (0.15) | 0.2740 | 0.5486 | −1.18 | ↓ |
| lysoPC a C26:1 | 0.27 (0.05) | 0.30 (0.07) | 0.6330 | 0.6420 | −1.09 | ↓ |
| lysoPC a C28:0 | 0.44 (0.10) | 0.55 (0.08) | 0.0120 | 0.1132 | −1.26 | ↓ |
| lysoPC a C28:1 | 0.38 (0.06) | 0.41 (0.08) | 0.3600 | 0.6066 | −1.09 | ↓ |
| PC aa C24:0 | 0.21 (0.05) | 0.20 (0.05) | 0.7620 | 0.8271 | 1.06 | ↑ |
| PC aa C26:0 | 1.71 (0.44) | 1.92 (0.39) | 0.2740 | 0.5992 | −1.12 | ↓ |
| PC aa C28:1 | 0.38 (0.10) | 0.43 (0.08) | 0.2370 | 0.5992 | −1.12 | ↓ |
| PC aa C30:0 | 0.78 (0.14) | 0.82 (0.10) | 0.4600 | 0.7533 | −1.04 | ↓ |
| PC aa C32:0 | 16.87 (2.05) | 18.79 (1.74) | 0.0270 | 0.1936 | −1.11 | ↓ |
| PC aa C32:1 | 5.49 (1.47) | 6.86 (2.57) | 0.3600 | 0.4499 | −1.25 | ↓ |
| PC aa C32:2 | 1.29 (0.23) | 1.58 (0.31) | 0.0550 | 0.1530 | −1.23 | ↓ |
| PC aa C32:3 | 0.21 (0.03) | 0.20 (0.02) | 0.5150 | 0.5992 | 1.07 | ↑ |
| PC aa C34:1 | 184.28 (46.07) | 189.47 (37.75) | 0.6960 | 0.9283 | −1.03 | ↓ |
| PC aa C34:2 | 525.46 (83.54) | 559.54 (62.15) | 0.3600 | 0.5992 | −1.06 | ↓ |
| PC aa C34:3 | 23.92 (4.25) | 26.91 (5.73) | 0.4080 | 0.5181 | −1.12 | ↓ |
| PC aa C34:4 | 0.71 (0.15) | 0.92 (0.21) | 0.0430 | 0.1258 | −1.29 | ↓ |
| PC aa C36:0 | 2.98 (0.74) | 3.06 (0.60) | 0.5150 | 0.9321 | −1.02 | ↓ |
| PC aa C36:1 | 43.06 (9.14) | 46.29 (6.86) | 0.2370 | 0.6346 | −1.08 | ↓ |
| PC aa C36:2 | 298.71 (72.94) | 343.31 (52.28) | 0.1010 | 0.4435 | −1.15 | ↓ |
| PC aa C36:3 | 115.69 (23.05) | 140.05 (21.58) | 0.0430 | 0.1530 | −1.21 | ↓ |
| PC aa C36:4 | 211.51 (28.43) | 233.04 (48.23) | 0.3600 | 0.5324 | −1.10 | ↓ |
| PC aa C36:5 | 11.89 (2.55) | 12.16 (3.33) | 0.8290 | 0.9321 | −1.02 | ↓ |
| PC aa C36:6 | 0.55 (0.10) | 0.60 (0.10) | 0.3600 | 0.6124 | −1.08 | ↓ |
| PC aa C38:0 | 2.90 (0.55) | 2.73 (0.23) | 0.7620 | 0.6136 | 1.06 | ↑ |
| PC aa C38:1 | 0.81 (0.10) | 0.73 (0.18) | 0.5730 | 0.5324 | 1.11 | ↑ |
| PC aa C38:3 | 37.59 (8.09) | 41.08 (6.61) | 0.3600 | 0.5992 | −1.09 | ↓ |
| PC aa C38:4 | 133.63 (19.85) | 136.33 (27.36) | 0.8290 | 0.9289 | −1.02 | ↓ |
| PC aa C38:5 | 43.81 (6.73) | 46.55 (11.93) | 0.4080 | 0.7366 | −1.06 | ↓ |
| PC aa C38:6 | 211.26 (32.16) | 197.65 (26.10) | 0.5150 | 0.5992 | 1.07 | ↑ |
| PC aa C40:2 | 0.43 (0.09) | 0.58 (0.10) | 0.0060 | 0.0561 | −1.34 | ↓ |
| PC aa C40:3 | 0.77 (0.20) | 0.96 (0.17) | 0.0550 | 0.1949 | −1.24 | ↓ |
| PC aa C40:4 | 3.20 (0.70) | 3.87 (0.72) | 0.1010 | 0.2210 | −1.21 | ↓ |
| PC aa C40:5 | 7.62 (1.49) | 7.65 (1.53) | 0.6330 | 0.9943 | −1.00 | ↓ |
| PC aa C40:6 | 67.66 (15.18) | 56.47 (8.23) | 0.1010 | 0.2498 | 1.20 | ↑ |
| PC aa C42:0 | 0.16 (0.03) | 0.15 (0.02) | 0.3150 | 0.5349 | 1.08 | ↑ |
| PC aa C42:1 | 0.15 (0.03) | 0.15 (0.03) | 0.7620 | 0.9943 | 1.00 | ↑ |
| PC aa C42:2 | 0.22 (0.04) | 0.29 (0.05) | 0.0090 | 0.0525 | −1.35 | ↓ |
| PC aa C42:4 | 0.30 (0.05) | 0.36 (0.05) | 0.0270 | 0.1132 | −1.20 | ↓ |
| PC aa C42:5 | 0.45 (0.07) | 0.46 (0.08) | 0.6960 | 0.8366 | −1.03 | ↓ |
| PC aa C42:6 | 1.33 (0.27) | 1.49 (0.28) | 0.2370 | 0.5283 | −1.12 | ↓ |
| PC ae C30:0 | 0.17 (0.03) | 0.19 (0.02) | 0.1730 | 0.4435 | −1.11 | ↓ |
| PC ae C30:1 | 0.17 (0.07) | 0.21 (0.07) | 0.3150 | 0.5992 | −1.19 | ↓ |
| PC ae C30:2 | 0.10 (0.03) | 0.11 (0.02) | 0.4080 | 0.6661 | −1.09 | ↓ |
| PC ae C32:1 | 0.99 (0.22) | 1.00 (0.11) | 0.8970 | 0.9321 | −1.02 | ↓ |
| PC ae C32:2 | 0.38 (0.10) | 0.45 (0.06) | 0.1220 | 0.4025 | −1.16 | ↓ |
| PC ae C34:0 | 0.60 (0.13) | 0.68 (0.11) | 0.1730 | 0.4565 | −1.13 | ↓ |
| PC ae C34:1 | 4.87 (0.61) | 4.87 (0.47) | 0.8290 | 0.9970 | −1.00 | ↓ |
| PC ae C34:2 | 5.69 (1.82) | 6.18 (1.27) | 0.4080 | 0.7226 | −1.09 | ↓ |
| PC ae C34:3 | 1.34 (0.42) | 1.59 (0.37) | 0.1730 | 0.4806 | −1.19 | ↓ |
| PC ae C36:0 | 0.64 (0.15) | 0.69 (0.13) | 0.4600 | 0.7355 | −1.07 | ↓ |
| PC ae C36:1 | 2.80 (0.42) | 2.89 (0.46) | 0.6960 | 0.8366 | −1.03 | ↓ |
| PC ae C36:2 | 11.74 (4.65) | 14.12 (3.14) | 0.2030 | 0.5204 | −1.20 | ↓ |
| PC ae C36:3 | 2.97 (0.68) | 3.23 (0.42) | 0.1460 | 0.5992 | −1.09 | ↓ |
| PC ae C36:4 | 5.68 (0.98) | 5.47 (0.55) | 0.5150 | 0.7847 | 1.04 | ↑ |
| PC ae C36:5 | 2.65 (0.79) | 2.56 (0.36) | 0.9650 | 0.9283 | 1.03 | ↑ |
| PC ae C38:0 | 5.05 (0.93) | 5.17 (1.00) | 0.5730 | 0.9283 | −1.02 | ↓ |
| PC ae C38:1 | 0.95 (0.21) | 1.12 (0.26) | 0.1010 | 0.4229 | −1.17 | ↓ |
| PC ae C38:2 | 7.59 (4.63) | 10.01 (3.31) | 0.1010 | 0.5204 | −1.32 | ↓ |
| PC ae C38:3 | 1.87 (0.42) | 2.16 (0.37) | 0.0680 | 0.2305 | −1.16 | ↓ |
| PC ae C38:4 | 6.01 (1.02) | 6.18 (0.84) | 0.6330 | 0.8767 | −1.03 | ↓ |
| PC ae C38:5 | 5.50 (0.74) | 5.41 (0.65) | 0.6330 | 0.9283 | 1.02 | ↑ |
| PC ae C38:6 | 4.82 (1.24) | 4.31 (0.36) | 0.3150 | 0.5204 | 1.12 | ↑ |
| PC ae C40:1 | ive amount | 5.77 (0.86) | 0.2370 | 0.5181 | −1.09 | ↓ |
| PC ae C40:2 | 0.83 (0.15) | 0.88 (0.14) | 0.2740 | 0.6912 | −1.06 | ↓ |
| PC ae C40:3 | 0.80 (0.17) | 0.81 (0.13) | 0.4600 | 0.6761 | −1.02 | ↓ |
| PC ae C40:4 | 2.64 (0.64) | 2.89 (0.42) | 0.2740 | 0.5992 | −1.10 | ↓ |
| PC ae C40:5 | 1.36 (0.20) | 1.38 (0.17) | 0.9650 | 0.9482 | −1.01 | ↓ |
| PC ae C40:6 | 4.38 (1.12) | 4.00 (0.31) | 0.5730 | 0.5992 | 1.10 | ↑ |
| PC ae C42:0 | 1.90 (0.24) | 1.75 (0.26) | 0.3150 | 0.4806 | 1.09 | ↑ |
| PC ae C42:1 | 0.93 (0.12) | 0.82 (0.13) | 0.1010 | 0.2382 | 1.14 | ↑ |
| PC ae C42:2 | 0.50 (0.07) | 0.61 (0.10) | 0.0340 | 0.0833 | −1.23 | ↓ |
| PC ae C42:3 | 1.37 (0.22) | 1.76 (0.38) | 0.0430 | 0.1132 | −1.28 | ↓ |
| PC ae C42:4 | 0.25 (0.05) | 0.28 (0.03) | 0.4080 | 0.5992 | −1.08 | ↓ |
| PC ae C42:5 | 0.79 (0.11) | 0.80 (0.06) | 0.5730 | 0.9342 | −1.01 | ↓ |
| PC ae C44:3 | 0.07 (0.01) | 0.07 (0.01) | 0.5730 | 0.9283 | −1.02 | ↓ |
| PC ae C44:4 | 0.11 (0.01) | 0.13 (0.02) | 0.0210 | 0.1132 | −1.19 | ↓ |
| PC ae C44:5 | 0.19 (0.03) | 0.21 (0.02) | 0.2740 | 0.5324 | −1.07 | ↓ |
| PC ae C44:6 | 0.16 (0.03) | 0.17 (0.02) | 0.5730 | 0.7181 | −1.05 | ↓ |
| SM (OH) C14:1 | 1.80 (0.60) | 1.78 (0.42) | 0.9650 | 0.9943 | 1.01 | ↑ |
| SM (OH) C16:1 | 0.42 (0.14) | 0.42 (0.08) | 0.9650 | 0.9943 | 1.00 | ↑ |
| SM (OH) C22:1 | 2.91 (0.70) | 2.27 (0.41) | 0.0550 | 0.1530 | 1.28 | ↑ |
| SM (OH) C22:2 | 2.44 (0.51) | 2.05 (0.23) | 0.1010 | 0.1949 | 1.19 | ↑ |
| SM (OH) C24:1 | 0.30 (0.08) | 0.23 (0.04) | 0.0680 | 0.1132 | 1.34 | ↑ |
| SM C16:0 | 28.33 (8.82) | 25.81 (4.01) | 0.9650 | 0.9943 | 1.10 | ↑ |
| SM C16:1 | 5.97 (1.29) | 5.53 (0.54) | 0.6330 | 0.5992 | 1.08 | ↑ |
| SM C18:0 | 2.19 (0.90) | 2.04 (0.32) | 1.0000 | 0.8216 | 1.07 | ↑ |
| SM C18:1 | 1.04 (0.37) | 0.91 (0.14) | 0.8290 | 0.5992 | 1.15 | ↑ |
| SM C20:2 | 0.10 (0.05) | 0.08 (0.03) | 0.5730 | 0.7601 | 1.32 | ↑ |
| SM C24:0 | 10.69 (1.74) | 10.53 (1.52) | 0.9650 | 0.9321 | 1.02 | ↑ |
| SM C24:1 | 21.68 (6.70) | 16.81 (2.12) | 0.3150 | 0.5992 | 1.29 | ↑ |
| H1 | 18520.71 (2822.10) | 18981.06 (1936.74) | 0.5150 | 0.8678 | −1.02 | ↓ |
| Alanine | 431.10 (66.56) | 537.62 (80.23) | 0.0090 | 0.0630 | −1.25 | ↓ |
| Arginine | 167.40 (31.49) | 166.25 (34.16) | 0.9650 | 0.9943 | 1.01 | ↑ |
| Asparagine | 38.55 (5.90) | 46.17 (5.93) | 0.0140 | 0.1132 | −1.20 | ↓ |
| Aspartate | 26.26 (4.80) | 23.10 (4.24) | 0.2740 | 0.4435 | 1.14 | ↑ |
| Citrulline | 44.10 (11.04) | 63.38 (13.72) | 0.0090 | 0.0525 | −1.44 | ↓ |
| Glutamine | 880.20 (84.30) | 909.00 (94.40) | 0.8290 | 0.7203 | −1.03 | ↓ |
| Glutamic acid | 90.99 (16.95) | 80.29 (14.09) | 0.2130 | 0.4499 | 1.13 | ↑ |
| Glycine | 234.70 (36.14) | 273.38 (26.72) | 0.0680 | 0.1132 | −1.16 | ↓ |
| Histidine | 71.04 (3.98) | 80.04 (8.65) | 0.0290 | 0.1132 | −1.13 | ↓ |
| Isoleucine | 114.25 (23.68) | 120.00 (21.52) | 0.4770 | 0.7847 | −1.05 | ↓ |
| Leucine | 253.90 (55.00) | 302.75 (54.03) | 0.0830 | 0.2472 | −1.19 | ↓ |
| Lysine | 281.90 (61.37) | 335.00 (74.76) | 0.1310 | 0.3581 | −1.19 | ↓ |
| Methionine | 50.14 (18.85) | 71.83 (16.49) | 0.0370 | 0.1132 | −1.43 | ↓ |
| Ornithine | 51.66 (10.95) | 66.46 (15.56) | 0.0620 | 0.1396 | −1.29 | ↓ |
| Phenylalanine | 111.16 (17.90) | 112.09 (10.83) | 1.0000 | 0.9636 | −1.01 | ↓ |
| Proline | 95.14 (36.23) | 139.09 (29.61) | 0.0230 | 0.1132 | −1.46 | ↓ |
| Serine | 125.78 (28.90) | 152.25 (17.83) | 0.0500 | 0.1541 | −1.21 | ↓ |
| Threonine | 160.50 (45.41) | 189.75 (27.95) | 0.2660 | 0.3933 | −1.18 | ↓ |
| Tryptophan | 94.36 (12.42) | 94.61 (15.04) | 1.0000 | 0.9943 | −1.00 | ↓ |
| Tyrosine | 83.98 (30.52) | 95.46 (24.38) | 0.3600 | 0.6186 | −1.14 | ↓ |
| Valine | 234.40 (48.01) | 243.00 (51.33) | 0.7560 | 0.8767 | −1.04 | ↓ |
| Acetylornithine | 0.61 (0.10) | 0.65 (0.10) | 0.5730 | 0.6136 | −1.07 | ↓ |
| Asymmetric | 0.67 (0.24) | 0.75 (0.09) | 0.6960 | 0.8678 | −1.13 | ↓ |
| dimethylarginine | | | | | | |
| alpha-Aminoadipic | 10.41 (2.00) | 10.42 (3.23) | 0.9650 | 0.9970 | −1.00 | ↓ |
| acid | | | | | | |
| Carnosine | 0.84 (0.11) | 0.78 (0.11) | 0.5150 | 0.7212 | 1.08 | ↑ |
| Creatinine | 9.57 (4.12) | 6.67 (1.60) | 0.0430 | 0.1670 | 1.44 | ↑ |
| Kynurenine | 1.23 (0.20) | 1.08 (0.32) | 0.1200 | 0.5204 | 1.14 | ↑ |
| Methionine | 2.20 (1.83) | 3.56 (0.90) | 0.0270 | 0.2384 | −1.62 | ↓ |
| sulfoxide | | | | | | |
| c4-OH-Pro | 0.55 (0.19) | 0.43 (0.01) | 0.0230 | 0.1132 | 1.27 | ↑ |
| t4-OH-Pro | 0.40 (0.09) | 0.37 (0.00) | 0.7880 | 0.9283 | 1.09 | ↑ |
| Putrescine | 1.36 (0.42) | 0.95 (0.14) | 0.0210 | 0.1132 | 1.43 | ↑ |
| Serotonin | 21.36 (3.39) | 22.64 (4.84) | 0.5940 | 0.7212 | −1.06 | ↓ |
| Spermidine | 2.09 (0.80) | 1.08 (0.24) | 0.0030 | 0.0525 | 1.94 | ↑ |
| Taurine | 364.90 (33.52) | 390.00 (34.67) | 0.2030 | 0.4025 | −1.07 | ↓ |

In Table 4: Bonferroni Correction for p-value is 0.0003; p-value calculated using the Wilcoxon Mann Whitney

A total of 56 metabolites showed significant concentration changes in TBI cases sampled at 4 h compared to 24 h after trauma (Table 5).

TABLE 5
Metabolite Concentrations Change Over Time in Serum.

| | TBI Timing | | | | | |
|---|---|---|---|---|---|---|
| | TBI (24 Hour) - | TBI (4 Hour) - | | q-value | Fold | Late/ |
| Metabolite | late (n = 5) | early (n = 5) | p-value | (FDR) | Change | Early |
| C0 | 26.34 (7.43) | 21.70 (2.86) | 0.3100 | 0.3461 | 1.21 | ↑ |
| C14 | 0.08 (0.02) | 0.10 (0.00) | 0.2220 | 0.3406 | −1.22 | ↓ |
| C14:1 | 0.10 (0.01) | 0.10 (0.01) | 1.0000 | 0.8602 | 1.02 | ↑ |
| C14:2 | 0.03 (0.01) | 0.03 (0.00) | 1.0000 | 0.9581 | −1.01 | ↓ |
| C16 | 0.22 (0.08) | 0.24 (0.02) | 0.6900 | 0.6860 | −1.09 | ↓ |
| C16:1 | 0.06 (0.02) | 0.07 (0.01) | 0.0950 | 0.2880 | −1.21 | ↓ |
| C18 | 0.06 (0.02) | 0.06 (0.01) | 1.0000 | 0.7937 | 1.05 | ↑ |
| C18:1 | 0.22 (0.08) | 0.21 (0.03) | 1.0000 | 0.8499 | 1.06 | ↑ |
| C18:2 | 0.10 (0.03) | 0.09 (0.01) | 0.5480 | 0.4208 | 1.16 | ↑ |
| C2 | 23.33 (3.90) | 27.97 (5.03) | 0.3100 | 0.4299 | −1.20 | ↓ |
| C3 | 1.13 (0.20) | 0.55 (0.08) | 0.0080 | 0.0073 | 2.05 | ↑ |
| C5—OH (C3-DC-M) | 0.07 (0.01) | 0.08 (0.00) | 0.0160 | 0.0529 | −1.14 | ↓ |
| C4 | 2.34 (0.81) | 2.01 (0.44) | 0.6900 | 0.5440 | 1.17 | ↑ |
| C3-DC (C4—OH) | 0.16 (0.05) | 0.16 (0.03) | 0.4210 | 0.5296 | −1.00 | ↓ |
| C5 | 0.30 (0.04) | 0.17 (0.02) | 0.0080 | 0.0022 | 1.76 | ↑ |
| C6 (C4:1-DC) | 0.24 (0.10) | 0.22 (0.05) | 1.0000 | 0.7158 | 1.12 | ↑ |
| lysoPC a C16:0 | 311.26 (44.67) | 303.39 (48.81) | 0.5480 | 0.8602 | 1.03 | ↑ |
| lysoPC a C16:1 | 5.35 (1.41) | 9.10 (2.19) | 0.0160 | 0.0483 | −1.70 | ↓ |
| lysoPC a C17:0 | 4.21 (0.53) | 3.26 (0.45) | 0.0160 | 0.0529 | 1.29 | ↑ |
| lysoPC a C18:0 | 133.27 (25.44) | 116.97 (21.55) | 0.3100 | 0.4295 | 1.14 | ↑ |
| lysoPC a C18:1 | 44.73 (8.41) | 59.98 (15.38) | 0.1510 | 0.1642 | −1.34 | ↓ |
| lysoPC a C18:2 | 158.54 (46.13) | 121.95 (20.68) | 0.2220 | 0.3406 | 1.30 | ↑ |
| lysoPC a C20:3 | 5.38 (2.13) | 7.56 (1.80) | 0.1510 | 0.2081 | −1.40 | ↓ |
| lysoPC a C20:4 | 33.72 (9.00) | 37.53 (2.59) | 0.6900 | 0.5296 | −1.11 | ↓ |
| lysoPC a C24:0 | 0.85 (0.08) | 0.68 (0.09) | 0.0560 | 0.0548 | 1.24 | ↑ |
| lysoPC a C26:0 | 0.57 (0.18) | 0.54 (0.09) | 0.8410 | 0.8825 | 1.06 | ↑ |
| lysoPC a C26:l | 0.27 (0.06) | 0.27 (0.03) | 0.8410 | 0.8991 | −1.02 | ↓ |
| lysoPC a C28:0 | 0.47 (0.13) | 0.40 (0.05) | 0.3100 | 0.3766 | 1.19 | ↑ |
| lysoPC a C28:1 | 0.41 (0.08) | 0.36 (0.04) | 0.4210 | 0.4005 | 1.13 | ↑ |
| PC aa C24:0 | 0.19 (0.04) | 0.23 (0.06) | 0.2220 | 0.3576 | −1.20 | ↓ |
| PC aa C26:0 | 1.96 (0.51) | 1.47 (0.18) | 0.1510 | 0.1501 | 1.34 | ↑ |
| PC aa C28:1 | 0.45 (0.12) | 0.32 (0.03) | 0.0560 | 0.1453 | 1.39 | ↑ |
| PC aa C30:0 | 0.89 (0.12) | 0.68 (0.03) | 0.0080 | 0.0529 | 1.32 | ↑ |
| PC aa C32:0 | 17.94 (1.85) | 15.81 (1.80) | 0.0950 | 0.1800 | 1.13 | ↑ |
| PC aa C32:1 | 4.51 (0.79) | 6.48 (1.34) | 0.0320 | 0.0669 | −1.44 | ↓ |
| PC aa C32:2 | 1.24 (0.25) | 1.33 (0.23) | 0.6900 | 0.6759 | −1.07 | ↓ |
| PC aa C32:3 | 0.24 (0.02) | 0.19 (0.01) | 0.0080 | 0.0213 | 1.28 | ↑ |
| PC aa C34:1 | 155.79 (20.33) | 212.77 (48.30) | 0.0320 | 0.1011 | −1.37 | ↓ |
| PC aa C34:2 | 589.48 (45.82) | 461.43 (57.94) | 0.0320 | 0.0307 | 1.28 | ↑ |
| PC aa C34:3 | 21.63 (3.77) | 26.22 (3.63) | 0.1510 | 0.1622 | −1.21 | ↓ |
| PC aa C34:4 | 0.63 (0.13) | 0.80 (0.12) | 0.0950 | 0.1244 | −1.28 | ↓ |
| PC aa C36:0 | 3.39 (0.81) | 2.58 (0.42) | 0.2220 | 0.1535 | 1.32 | ↑ |
| PC aa C36:1 | 42.39 (6.28) | 43.73 (12.15) | 1.0000 | 0.8825 | −1.03 | ↓ |
| PC aa C36:2 | 350.93 (64.94) | 246.48 (30.56) | 0.0320 | 0.0483 | 1.42 | ↑ |
| PC aa C36:3 | 122.16 (31.77) | 109.21 (9.03) | 0.6900 | 0.5296 | 1.12 | ↑ |
| PC aa C36:4 | 219.72 (28.79) | 203.29 (28.66) | 0.4210 | 0.5251 | 1.08 | ↑ |
| PC aa C36:5 | 12.85 (3.09) | 10.94 (1.67) | 0.3100 | 0.3783 | 1.17 | ↑ |
| PC aa C36:6 | 0.53 (0.11) | 0.58 (0.10) | 0.5480 | 0.5296 | −1.11 | ↓ |
| PC aa C38:0 | 3.23 (0.50) | 2.57 (0.41) | 0.0560 | 0.1173 | 1.26 | ↑ |
| PC aa C38:1 | 0.77 (0.13) | 0.85 (0.06) | 0.3100 | 0.3406 | −1.11 | ↓ |
| PC aa C38:3 | 35.77 (8.55) | 39.41 (8.12) | 0.5480 | 0.6159 | −1.10 | ↓ |
| PC aa C38:4 | 134.76 (22.62) | 132.50 (19.27) | 1.0000 | 0.8991 | 1.02 | ↑ |
| PC aa C38:5 | 39.22 (5.53) | 48.39 (4.31) | 0.0320 | 0.0588 | −1.23 | ↓ |
| PC aa C38:6 | 217.81 (42.17) | 204.71 (21.02) | 0.8410 | 0.6552 | 1.06 | ↑ |
| PC aa C40:2 | 0.44 (0.12) | 0.42 (0.07) | 0.6900 | 0.8602 | 1.04 | ↑ |
| PC aa C40:3 | 0.65 (0.23) | 0.89 (0.07) | 0.0950 | 0.1535 | −1.37 | ↓ |
| PC aa C40:4 | 2.90 (0.68) | 3.50 (0.64) | 0.1510 | 0.2958 | −1.21 | ↓ |
| PC aa C40:5 | 7.07 (0.80) | 8.18 (1.90) | 0.4210 | 0.5296 | −1.16 | ↓ |
| PC aa C40:6 | 78.56 (11.91) | 56.76 (8.91) | 0.0160 | 0.0483 | 1.38 | ↑ |
| PC aa C42:0 | 0.18 (0.03) | 0.14 (0.02) | 0.0320 | 0.1028 | 1.23 | ↑ |
| PC aa C42:1 | 0.16 (0.02) | 0.13 (0.01) | 0.0560 | 0.0799 | 1.25 | ↑ |
| PC aa C42:2 | 0.24 (0.05) | 0.19 (0.02) | 0.0950 | 0.1721 | 1.22 | ↑ |
| PC aa C42:4 | 0.31 (0.05) | 0.29 (0.05) | 0.8410 | 0.6860 | 1.06 | ↑ |
| PC aa C42:5 | 0.45 (0.09) | 0.45 (0.04) | 0.8410 | 1.0000 | 1.00 | ↑ |
| PC aa C42:6 | 1.32 (0.27) | 1.35 (0.31) | 1.0000 | 1.0000 | −1.02 | ↓ |
| PC ae C30:0 | 0.19 (0.03) | 0.15 (0.02) | 0.0560 | 0.1264 | 1.23 | ↑ |
| PC ae C30:1 | 0.21 (0.08) | 0.13 (0.03) | 0.0950 | 0.1244 | 1.60 | ↑ |
| PC ae C30:2 | 0.12 (0.03) | 0.08 (0.01) | 0.0560 | 0.0908 | 1.41 | ↑ |
| PC ae C32:1 | 1.15 (0.15) | 0.83 (0.16) | 0.0320 | 0.0483 | 1.39 | ↑ |
| PC ae C32:2 | 0.45 (0.09) | 0.31 (0.03) | 0.0160 | 0.0756 | 1.44 | ↑ |
| PC ae C34:0 | 0.69 (0.11) | 0.50 (0.05) | 0.0160 | 0.0411 | 1.38 | ↑ |
| PC ae C34:1 | 5.16 (0.61) | 4.58 (0.50) | 0.2220 | 0.2329 | 1.13 | ↑ |
| PC ae C34:2 | 7.14 (1.39) | 4.24 (0.53) | 0.0080 | 0.0213 | 1.68 | ↑ |
| PC ae C34:3 | 1.58 (0.48) | 1.10 (0.11) | 0.1510 | 0.2459 | 1.44 | ↑ |
| PC ae C36:0 | 0.77 (0.10) | 0.52 (0.06) | 0.0080 | 0.0126 | 1.49 | ↑ |
| PC ae C36:1 | 2.99 (0.43) | 2.62 (0.35) | 0.1510 | 0.2718 | 1.14 | ↑ |
| PC ae C36:2 | 15.27 (4.03) | 8.21 (1.15) | 0.0080 | 0.0529 | 1.86 | ↑ |
| PC ae C36:3 | 3.43 (0.66) | 2.51 (0.24) | 0.0320 | 0.0582 | 1.37 | ↑ |
| PC ae C36:4 | 6.32 (0.56) | 5.03 (0.89) | 0.0320 | 0.0739 | 1.26 | ↑ |
| PC ae C36:5 | 3.31 (0.43) | 1.98 (0.30) | 0.0080 | 0.0073 | 1.68 | ↑ |
| PC ae C38:0 | 4.55 (1.02) | 5.55 (0.54) | 0.1510 | 0.1650 | −1.22 | ↓ |
| PC ae C38:1 | 1.09 (0.21) | 0.82 (0.09) | 0.0320 | 0.0756 | 1.34 | ↑ |
| PC ae C38:2 | 10.52 (5.13) | 4.66 (0.62) | 0.0320 | 0.0807 | 2.26 | ↑ |
| PC ae C38:3 | 2.12 (0.48) | 1.62 (0.09) | 0.0160 | 0.0529 | 1.31 | ↑ |
| PC ae C38:4 | 6.59 (0.95) | 5.43 (0.78) | 0.0560 | 0.1369 | 1.21 | ↑ |
| PC ae C38:5 | 5.94 (0.73) | 5.06 (0.47) | 0.0950 | 0.1184 | 1.17 | ↑ |
| PC ae C38:6 | 5.71 (1.05) | 3.94 (0.61) | 0.0160 | 0.0483 | 1.45 | ↑ |
| PC ae C40:1 | 5.42 (0.84) | 5.16 (0.71) | 0.6900 | 0.6977 | 1.05 | ↑ |
| PC ae C40:2 | 0.93 (0.15) | 0.74 (0.08) | 0.0320 | 0.1028 | 1.26 | ↑ |
| PC ae C40:3 | 0.86 (0.22) | 0.73 (0.08) | 0.4210 | 0.5296 | 1.18 | ↑ |
| PC ae C40:4 | 2.97 (0.71) | 2.31 (0.36) | 0.1510 | 0.1785 | 1.29 | ↑ |
| PC ae C40:5 | 1.50 (0.19) | 1.23 (0.10) | 0.0950 | 0.0686 | 1.22 | ↑ |
| PC ae C40:6 | 5.18 (0.94) | 3.58 (0.59) | 0.0160 | 0.0483 | 1.45 | ↑ |
| PC ae C42:0 | 2.06 (0.18) | 1.75 (0.18) | 0.0560 | 0.0790 | 1.17 | ↑ |
| PC ae C42:1 | 1.02 (0.08) | 0.84 (0.09) | 0.0320 | 0.0433 | 1.22 | ↑ |
| PC ae C42:2 | 0.45 (0.06) | 0.54 (0.03) | 0.0160 | 0.0529 | −1.20 | ↓ |
| PC ae C42:3 | 1.24 (0.23) | 1.50 (0.13) | 0.0950 | 0.1302 | −1.21 | ↓ |
| PC ae C42:4 | 0.30 (0.02) | 0.21 (0.01) | 0.0080 | 0.0007 | 1.47 | ↑ |
| PC ae C42:5 | 0.88 (0.07) | 0.70 (0.03) | 0.0080 | 0.0098 | 1.26 | ↑ |
| PC ae C44:3 | 0.08 (0.01) | 0.07 (0.01) | 0.0950 | 0.1476 | 1.19 | ↑ |
| PC ae C44:4 | 0.11 (0.01) | 0.10 (0.01) | 0.0950 | 0.1131 | 1.18 | ↑ |
| PC ae C44:5 | 0.22 (0.02) | 0.17 (0.01) | 0.0080 | 0.0218 | 1.24 | ↑ |
| PC ae C44:6 | 0.18 (0.02) | 0.14 (0.01) | 0.0160 | 0.0242 | 1.28 | ↑ |
| SM (OH) C14:1 | 2.30 (0.38) | 1.30 (0.20) | 0.0080 | 0.0100 | 1.78 | ↑ |
| SM (OH) C16:1 | 0.54 (0.08) | 0.30 (0.04) | 0.0080 | 0.0073 | 1.79 | ↑ |
| SM (OH) C22:1 | 3.37 (0.61) | 2.44 (0.42) | 0.0160 | 0.0673 | 1.38 | ↑ |
| SM (OH) C22:2 | 2.85 (0.36) | 2.02 (0.19) | 0.0080 | 0.0207 | 1.41 | ↑ |
| SM (OH) C24:1 | 0.37 (0.06) | 0.24 (0.03) | 0.0080 | 0.0172 | 1.59 | ↑ |
| SM C16:0 | 36.46 (2.91) | 20.20 (1.14) | 0.0080 | 0.0411 | 1.80 | ↑ |
| SM C16:1 | 7.16 (0.24) | 4.78 (0.39) | 0.0080 | 0.0411 | 1.50 | ↑ |
| SM C18:0 | 3.01 (0.35) | 1.36 (0.11) | 0.0080 | 0.0411 | 2.21 | ↑ |
| SM C18:1 | 1.38 (0.15) | 0.71 (0.07) | 0.0080 | 0.0010 | 1.95 | ↑ |
| SM C20:2 | 0.14 (0.05) | 0.07 (0.02) | 0.0560 | 0.1244 | 1.83 | ↑ |
| SM C24:0 | 11.81 (1.25) | 9.56 (1.44) | 0.0950 | 0.0797 | 1.23 | ↑ |
| SM C24:1 | 27.65 (2.99) | 15.72 (1.76) | 0.0080 | 0.0022 | 1.76 | ↑ |
| H1 | 20744.71 (2253.91) | 16296.72 (688.53) | 0.0080 | 0.0453 | 1.27 | ↑ |
| Alanine | 477.60 (60.83) | 384.60 (29.36) | 0.0120 | 0.0529 | 1.24 | ↑ |
| Arginine | 189.20 (24.01) | 145.60 (21.59) | 0.0590 | 0.0541 | 1.30 | ↑ |
| Asparagine | 41.46 (6.21) | 35.64 (4.32) | 0.1510 | 0.2109 | 1.16 | ↑ |
| Aspartate | 27.50 (4.19) | 25.02 (5.52) | 0.5480 | 0.5447 | 1.10 | ↑ |
| Citrulline | 52.40 (8.32) | 35.80 (5.74) | 0.0320 | 0.0393 | 1.46 | ↑ |
| Glutamine | 848.40 (113.97) | 912.00 (21.76) | 0.4210 | 0.4047 | −1.07 | ↓ |
| Glutamic acid | 82.04 (11.75) | 99.94 (17.56) | 0.1510 | 0.1721 | −1.22 | ↓ |
| Glycine | 242.00 (42.68) | 227.40 (31.36) | 0.6000 | 0.6552 | 1.06 | ↑ |
| Histidine | 69.84 (4.01) | 72.24 (3.98) | 0.5300 | 0.5049 | −1.03 | ↓ |
| Isoleucine | 108.08 (15.31) | 120.42 (30.53) | 0.8340 | 0.5440 | −1.11 | ↓ |
| Leucine | 269.80 (39.05) | 238.00 (68.19) | 0.3460 | 0.5251 | 1.13 | ↑ |
| Lysine | 329.20 (45.68) | 234.60 (28.18) | 0.0320 | 0.0292 | 1.40 | ↑ |
| Methionine | 66.20 (11.23) | 34.08 (5.33) | 0.0120 | 0.0073 | 1.94 | ↑ |
| Ornithine | 54.00 (7.73) | 49.32 (14.01) | 0.3100 | 0.6378 | 1.09 | ↑ |
| Phenylalanine | 120.00 (12.35) | 102.32 (19.31) | 0.2220 | 0.2109 | 1.17 | ↑ |
| Proline | 125.54 (25.12) | 64.74 (3.46) | 0.0120 | 0.0483 | 1.94 | ↑ |
| Serine | 148.20 (22.06) | 103.36 (11.67) | 0.0160 | 0.0275 | 1.43 | ↑ |
| Threonine | 202.00 (14.71) | 119.00 (10.82) | 0.0120 | 0.0007 | 1.70 | ↑ |
| Tryptophan | 98.36 (13.98) | 90.36 (10.58) | 0.5480 | 0.4643 | 1.09 | ↑ |
| Tyrosine | 109.50 (20.03) | 58.46 (8.15) | 0.0080 | 0.0100 | 1.87 | ↑ |
| Valine | 227.00 (14.47) | 241.80 (69.58) | 0.6740 | 0.7487 | −1.07 | ↓ |
| Acetylornithine | 0.62 (0.10) | 0.61 (0.11) | 0.8410 | 0.9121 | 1.02 | ↑ |
| Asymmetric | 0.56 (0.31) | 0.77 (0.09) | 0.1510 | 0.2459 | −1.36 | ↓ |
| dimethylarginine | | | | | | |
| alpha- | 11.16 (2.13) | 9.66 (1.74) | 0.2220 | 0.3783 | 1.15 | ↑ |
| Aminoadipic acid | | | | | | |
| Carnosine | 0.83 (0.12) | 0.85 (0.10) | 0.8410 | 0.8825 | −1.03 | ↓ |
| Creatinine | 9.42 (4.16) | 9.73 (4.56) | 0.6900 | 0.7729 | −1.03 | ↓ |
| Kynurenine | 1.18 (0.19) | 1.29 (0.22) | 0.2950 | 0.5296 | −1.10 | ↓ |
| Methionine | 3.48 (1.82) | 0.91 (0.24) | 0.0080 | 0.0411 | 3.84 | ↑ |
| sulfoxide | | | | | | |
| c4-OH-Pro | 0.52 (0.20) | 0.57 (0.20) | 0.8340 | 0.8825 | −1.09 | ↓ |
| t4-OH-Pro | 0.42 (0.13) | 0.38 (0.04) | 0.9160 | 0.9342 | 1.10 | ↑ |
| Putrescine | 1.60 (0.42) | 1.11 (0.27) | 0.0950 | 0.1244 | 1.45 | ↑ |
| Serotonin | 19.72 (3.20) | 23.00 (2.97) | 0.2220 | 0.2224 | −1.17 | ↓ |
| Spermidine | 2.72 (0.55) | 1.46 (0.35) | 0.0080 | 0.0213 | 1.87 | ↑ |
| Taurine | 374.40 (38.07) | 355.40 (29.22) | 0.4210 | 0.5296 | 1.05 | ↑ |

In Table 5: Bonferroni Correction for p-value is 0.0003; p-value was calculated by the Wilcoxon Mann Whitney test

Weight is a potential confounder so the inventors compared the weight of the TBI and control mice. There was no significant difference in the mean (SD) body weight 25.1 (1.93) g versus 24.7 (1.90) g, p value=0.624. The weight differences between cases undergoing sampling at 4 and at 24 h were also not significant (p=1.0). FIG. 6 displays a heat map which demonstrates the differences in metabolite concentrations in the TBI mice compared to control mice.

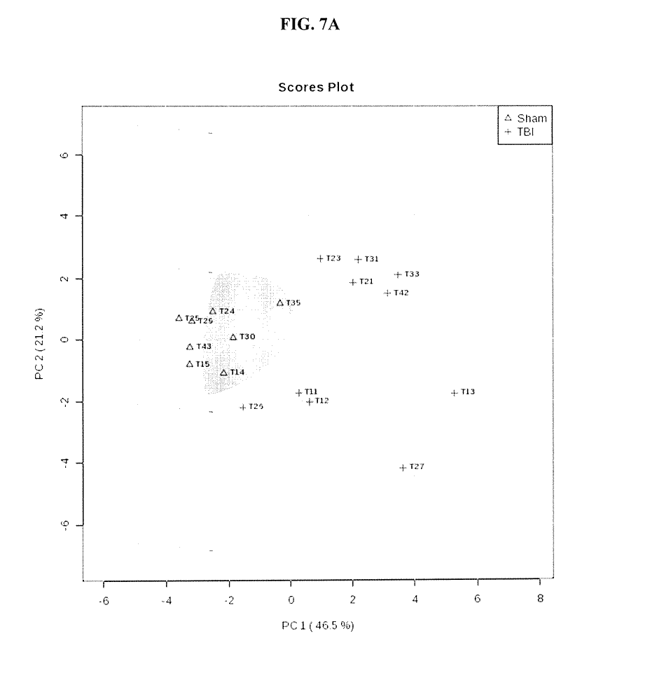

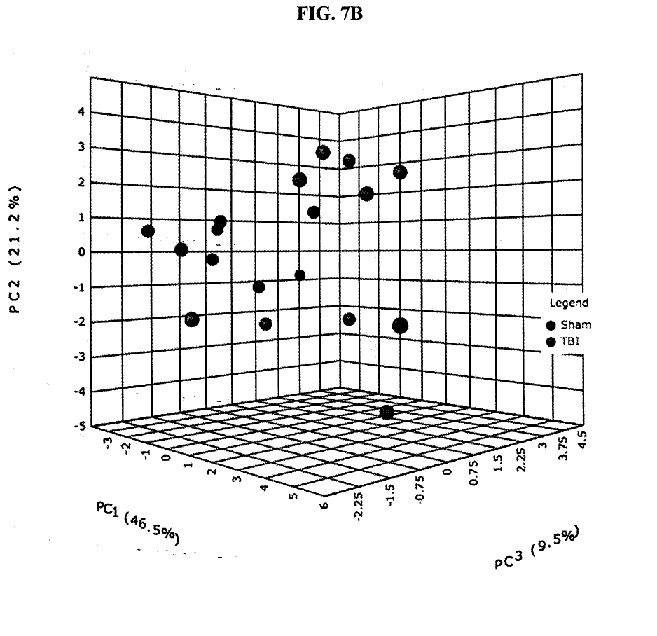

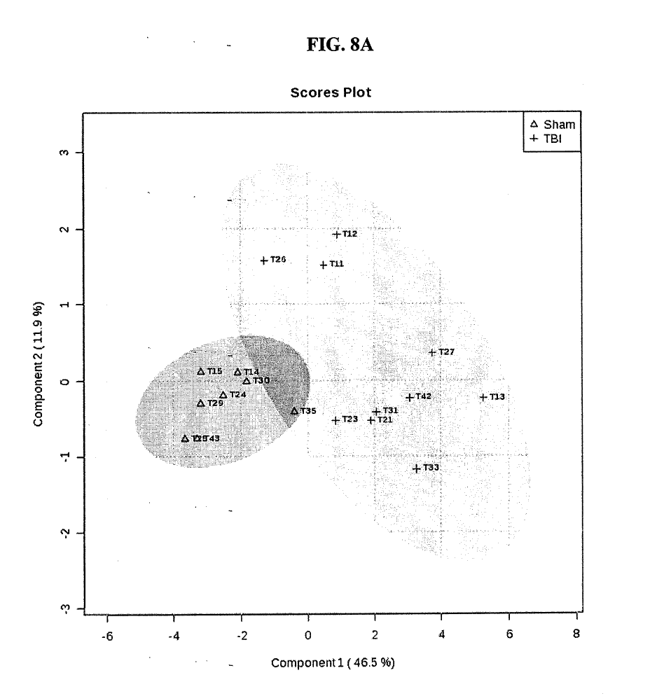

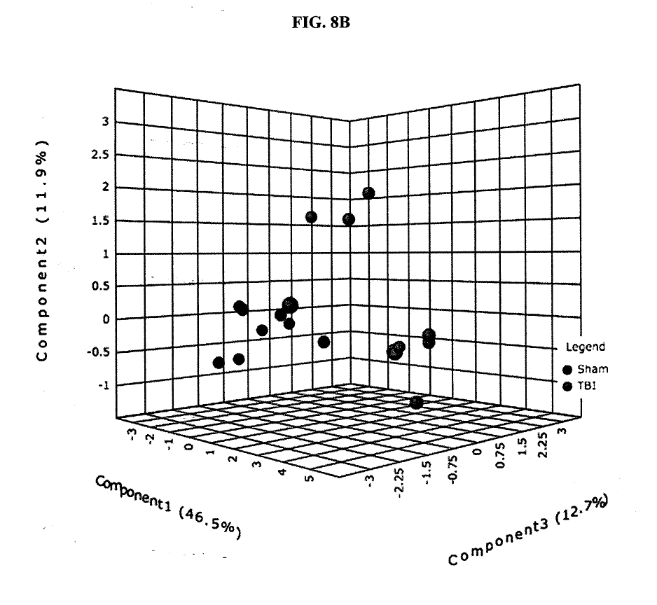

FIGS. 7A-7B and 8A-8B show the PCA and the PLS-DA score plots for TBI versus controls, respectively. In addition, FIG. 9 displays the predictive accuracy following cross-validation. Clear segregation of the two groups was achieved based on the metabolites.

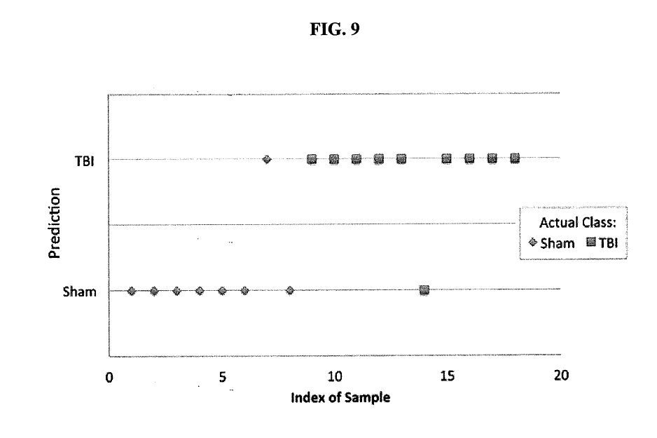

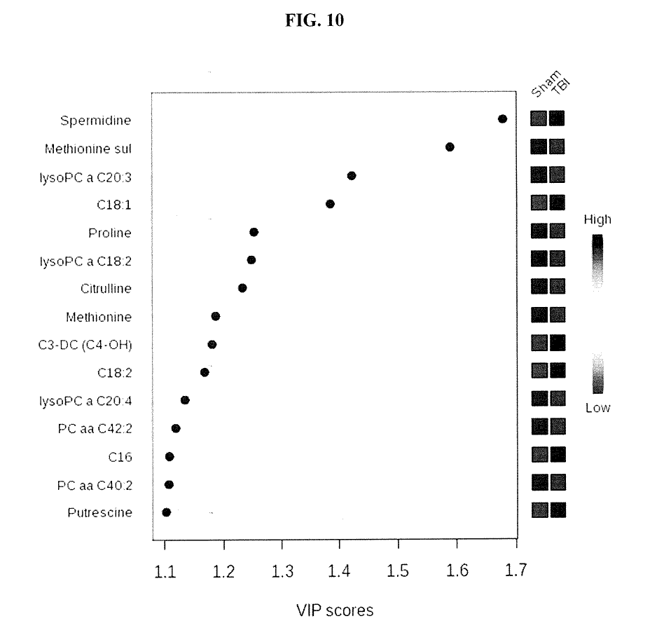

FIG. 10 shows the VIP plots for TBI mice versus controls. The discriminating metabolites are ranked in order of importance based on (increasing) VIP scores. For the generated PLS-DA model using a significant subset of metabolites (p value <0.3 calculated from the univariate analysis), permutation testing using 2000 repeats was performed to ascertain whether the separation achieved between cases and controls was likely due to chance. A p value=0.0025 was determined, indicating that the observed separation was not due to chance. The inventors also investigated whether significant metabolite differences developed over time post TBI. They found that metabolites were able to accurately characterize the interval after TBI or the acuteness of the TBI. Both the 2D and 3D PCA and PLS-DA analyses distinguished early (4 h) versus late (24 h) TBI (not shown).

The metabolites that were most useful in discriminating TBI from sham-operated controls are shown in the VIP plot (FIG. 10). For the generated PLS-DA model, permutation testing with 2000 repetitions revealed that the observed separation were unlikely to be due to chance, p value=0.024. Based on logistic regression analysis, a model for the prediction of TBI was developed, (see, Table 6).

TABLE 6
Predictive Models for the detection of TBI (overall)
versus Controls (sham-operated): Serum metabolites

| | | | Std. | | | |
|---|---|---|---|---|---|---|
| Model^{a} | Coefficients^{b} | Estimate | Error | z value | Pr(>\|z\|) | Odds Ratio |
| Demographics | | — | | | | |
| only | (Intercept) | −3.219 | 6.584 | −0.489 | 0.625 | — |
| | Weight | 0.138 | 0.264 | 0.524 | 0.600 | 1.15 (0.68-2.00) |

| | | | Std. | | | | TBI/ |
|---|---|---|---|---|---|---|---|
| Model^{b} | Coefficients^{b} | Estimate | Error | z value | Pr(>\|z\|) | Odds Ratio | Normal |
| Metabolites | (Intercept) | 1.106 | 1.507 | 0.734 | 0.463 | — | — |
| only | PC aa C34:4 | −4.450 | 2.431 | −1.831 | 0.067 | 0.01 (0.0-0.52) | Down |
| | Methionine | −1.883 | 0.940 | −2.003 | 0.045 | 0.15 (0.01-0.67) | Down |
| | sulfoxide | | | | | | |

| | | | Std. | | | | TBI/ |
|---|---|---|---|---|---|---|---|
| Model^{c} | Coefficients^{c} | Estimate | Error | z value | Pr(>\|z\|) | Odds Ratio | Normal |
| Metabolites | (Intercept) | 2.030 | 1.372 | 1.480 | 0.139 | — | — |
| combined | Interval | 2.831 | 1.949 | 1.452 | 0.146 | 16.96 (0.78-3128.9) | — |
| with | Time | | | | | | |
| Demographics | (=24 Hr) | | | | | | |
| | Methionine | −2.617 | 1.239 | −2.113 | 0.035 | 0.07 (0.0-0.47) | Down |
| | sulfoxide | | | | | | |

Note:
In this data analysis, Interval Time is unavailable to make model, even though this could be a significant feature.

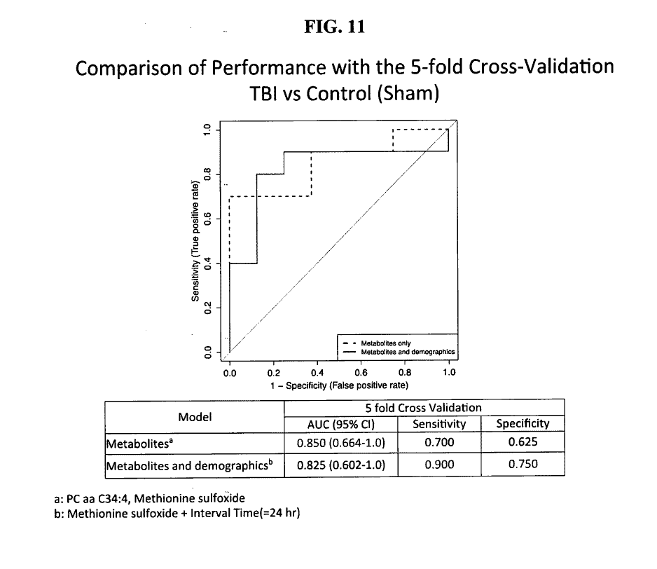

The combination of two metabolites, the lipid PC aa C 34:4 and the amino acid methionine sulfoxide yielded excellent performance for TBI detection with an AUC=0.85 (CI 0.66-1.0) following a five-fold CV (see, Table 7 and FIG. 11).

Table 7: Performance of Predictive Models for the detection of TBI (overall) versus Controls (sham-operated): Serum metabolites.

Demographics only - algorithm^{a}

| Discovery Data | | | 5 fold Cross Validation | | |
|---|---|---|---|---|---|
| AUC | Sensi- | | AUC | Sensi- | |
| (95% CI) | tivity | Specificity | (95% CI) | tivity | Specificity |
| 0.613 | 0.775 | 0.312 | 0.738) | 0.400 | 0.125 |
| (0.482-0.744) | | | (0.490-0.985 | | |

Metabolites only - algorithm^{b}

| Discovery Data | | | 5 fold Cross Validation | | |
|---|---|---|---|---|---|
| AUC | Sensi- | | AUC | Sensi- | |
| (95% CI) | tivity | Specificity | (95% CI) | tivity | Specificity |
| 0.965 | 0.825 | 0.750 | 0.850 | 0.700 | 0.625 |
| (0.871-0.981) | | | (0.664-1.0) | | |

Combined metabolites and demographics - algorithm^{c}

| Discovery Data | | | 5 fold Cross Validation | | |
|---|---|---|---|---|---|
| AUC | Sensi- | | AUC | Sensi- | |
| (95% CI) | tivity | Specificity | (95% CI) | tivity | Specificity |
| 0.903 | 0.875 | 0.750 | 0.825 | 0.900 | 0.750 |
| (0.835-0.972) | | | (0.602-1.0) | | |

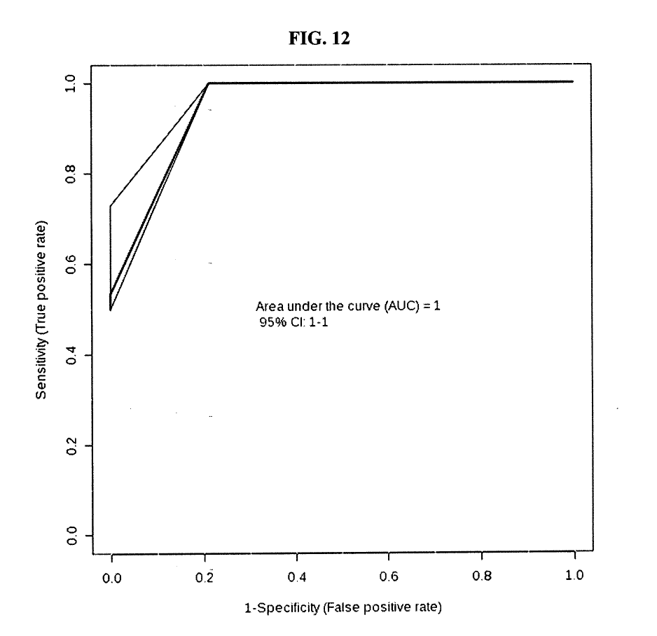

In addition to identifying serum biomarkers that could distinguish TBI from controls, the inventors were also interested in determining whether metabolite biomarkers could be used to determine the timing or chronicity of TBI. Due to the small number of samples in the early versus late TBI categories, ROC analysis could not be performed with the logistic regression approach. Using the Biomarker Analysis module of the MetaboAnalyst Program Xia et al. (2009, 2012, 2015), ROC analysis was performed with the PLS-DA model that was generated using the six most significantly different metabolites. As shown in FIG. 12, the combination of six metabolites: methionine sulfoxide, C3, SM C18:0, SM C 18:1, methionine and proline, achieved complete accuracy for distinguishing early TBI (4 h) from late TBI (24 h) with an AUC (95% CI)=1.0 (1.0-1.0). A p value=0.007 of permutation testing (2000 repetitions) indicates that the observed separation was not due to chance.

Pathway Analysis (Table 8) indicated a significant disturbance in several metabolic pathways as a result of TBI. Of these, four biochemical pathways: arginine and proline metabolism, glutathione metabolism, cysteine metabolism, and sphingolipid metabolism (in the later Holm p value=0.05) achieved statistical significance based on stringent criteria, Holm adjusted p value <0.05 and q-value (FDR)<0.05.

TABLE 8
Pathway analyses TBI demonstrating biochemical pathways
affected in the serum of TBI (combined) cases

| | Total | | | | | | |
|---|---|---|---|---|---|---|---|
| | Compounds | Hits | Raw p | −log (p) | Holm p | FDR | Impact |
| Arginine and proline | 77 | 6 | 5.61E−05 | 9.7892 | 0.0015695 | 0.0015695 | 0.33542 |
| metabolism | | | | | | | |
| Glutathione | 38 | 4 | 0.000339 | 7.991 | 0.0091398 | 0.0047392 | 0.05322 |
| metabolism | | | | | | | |
| Cysteine and | 56 | 3 | 0.001671 | 6.3941 | 0.043454 | 0.014048 | 0.05003 |
| methionine | | | | | | | |
| metabolism | | | | | | | |
| Sphingolipid | 25 | 2 | 0.002007 | 6.2112 | 0.050172 | 0.014048 | 0.00954 |
| metabolism | | | | | | | |
| beta-Alanine | 28 | 2 | 0.002995 | 5.8108 | 0.07188 | 0.016772 | 0.06625 |
| metabolism | | | | | | | |
| Glycerophospholipid | 39 | 2 | 0.005437 | 5.2146 | 0.12505 | 0.021109 | 0.1037 |
| metabolism | | | | | | | |
| Sulfur metabolism | 18 | 1 | 0.005503 | 5.2025 | 0.12505 | 0.021109 | 0 |
| Aminoacyl-tRNA | 75 | 8 | 0.006031 | 5.1108 | 0.12665 | 0.021109 | 0.05634 |
| biosynthesis | | | | | | | |
| Glycine, serine and | 48 | 2 | 0.01504 | 4.197 | 0.30081 | 0.039817 | 0.32378 |
| threonine | | | | | | | |
| metabolism | | | | | | | |
| Methane | 34 | 2 | 0.01504 | 4.197 | 0.30081 | 0.039817 | 0.01751 |
| metabolism | | | | | | | |
| Taurine and | 20 | 1 | 0.017064 | 4.0708 | 0.30716 | 0.039817 | 0.03237 |
| hypotaurine | | | | | | | |
| metabolism | | | | | | | |
| Selenoamino acid | 22 | 1 | 0.017064 | 4.0708 | 0.30716 | 0.039817 | 0 |
| metabolism | | | | | | | |
| Cyanoamino acid | 16 | 3 | 0.021599 | 3.8351 | 0.34559 | 0.046522 | 0 |
| metabolism | | | | | | | |
| Alanine, aspartate | 24 | 2 | 0.032151 | 3.4373 | 0.48227 | 0.064303 | 0.10256 |
| and glutamate | | | | | | | |
| metabolism | | | | | | | |

What is claimed is:

1. A method of detecting a level of two or more metabolites in a biological sample, said method consisting of:
obtaining a biological sample from a human patient, wherein said biological sample includes two or more of methionine sulfoxide, PC aa C 34:4, ATP, AMP, NAD+, ADP, IMP, spermidine, lysoPC a C20:3, C18:1, and proline; and
detecting the level of the two or more metabolites in the biological sample.

2. The method of claim 1, wherein the sample is blood serum.

3. The method of claim 2, wherein the two or more metabolites are methionine sulfoxide and PC aa C 34:4.

4. The method of claim 2, wherein the two or more metabolites are methionine sulfoxide, spermidine, lysoPC a C20:3, C18:1, and proline

5. The method of claim 1, wherein the two or more metabolites are ATP, AMP, ADP, and IMP.

6. The method of claim 1, wherein the two or more metabolites are AMP, NAD+, ADP, and IMP.

7. The method of claim 1, wherein the two or more metabolites are ATP, AMP, NAD+, ADP, and IMP.

8. The method of claim 1, wherein the level of the two or more metabolites are detected by performing Magnetic Resonance spectroscopy (MRS) using a magnetic resonance imaging (MRI) device, nuclear magnetic-resonance (NMR), or mass spectrometry (MS) on the biological sample.

9. The method of claim 8 wherein the two or more metabolites are detected by magnetic resonance spectroscopy (MRS).

10. The method of claim 9, wherein the MRS is proton magnetic resonance spectroscopy (1H-MRS)

11. A method of diagnosing traumatic brain injury (TBI) in a human patient, wherein said human patient has a brain, said method comprising:
obtaining a biological sample from the human patient, wherein said biological sample includes one or more metabolites methionine sulfoxide, PC aa C 34:4, ATP, AMP, NAD+, ADP, IMP, spermidine, lysoPC a C20:3, C18:1, and proline;
detecting a level of the one or more metabolites in the biological sample;
performing computerized tomography (CT) scanning, magnetic resonance imaging (MRI), or positron emission tomography (PET) on the brain of the patient; and
diagnosing the patient with traumatic brain injury when (a) the one or more metabolites in the biological sample is at a different level than a statistically validated threshold for the one or more metabolites and (b) the CT, MRI, or PET indicates TBI to the brain of the patient.

12. The method of claim 11, wherein the level of the one or more metabolites are detected by performing Magnetic Resonance spectroscopy (MRS) using a magnetic resonance imaging (MRI) device, nuclear magnetic resonance (NMR), or mass spectrometry (MS) on the biological sample.

13. The method of claim 12 wherein the one or more metabolites are detected by magnetic resonance spectroscopy (MRS).

14. The method of claim 13, wherein the MRS is proton magnetic resonance spectroscopy (1H-MRS)

15. The method of claim 11, wherein the sample is blood serum.

16. The method of claim 15, wherein the one or more metabolites are methionine sulfoxide and PC aa C 34:4.

17. The method of claim 15, wherein the one or more metabolites are methionine sulfoxide, spermidine, lysoPC a C20:3, C18:1, and proline

18. The method of claim 11, wherein the one or more metabolites are ATP, AMP, ADP, and IMP.

19. The method of claim 11, wherein the one or more metabolites are AMP, NAD+, ADP, and IMP.

20. The method of claim 11, wherein the one or more metabolites are ATP, AMP, NAD+, ADP, and IMP.

21. A method of diagnosing and treating TBI in a subject, said method comprising:
obtaining a biological sample from the human subject, wherein said biological sample includes one or more metabolites methionine sulfoxide, PC aa C 34:4, ATP, AMP, NAD+, ADP, IMP, spermidine, lysoPC a C20:3, C18:1, and proline;
detecting a level of the one or more metabolites in the biological sample;
diagnosing the subject with traumatic brain injury when the one or more metabolites in the biological sample is at a different level than a statistically validated threshold for the one or more metabolites; and
administering a therapeutically effective amount of a treatment for TBI to the diagnosed subject.

22. The method of claim 21, wherein the treatment is side-lining the subject.

23. The method of claim 22, wherein the subject is side-lined for about 1-2 hours.

24. The method of claim 21, wherein the level of the one or more metabolites are detected by performing Magnetic Resonance spectroscopy (MRS) using a magnetic resonance imaging (MRI) device, nuclear magnetic resonance (NMR), or mass spectrometry (MS) on the biological sample.

25. The method of claim 24 wherein the one or more metabolites are detected by magnetic resonance spectroscopy (MRS).

26. The method of claim 25, wherein the MRS is proton magnetic resonance spectroscopy (1H-MRS)

27. The method of claim 21, wherein the sample is blood serum.

28. The method of claim 27, wherein the one or more metabolites are methionine sulfoxide and PC aa C 34:4.

29. The method of claim 27, wherein the one or more metabolites are methionine sulfoxide, spermidine, lysoPC a C20:3, C18:1, and proline.

30. The method of claim 21; wherein the one or more metabolites are ATP, AMP, ADP, and IMP.

31. The method of claim 21, wherein the one or more metabolites are AMP, NAD+, ADP, and IMP.

32. The method of claim 21, wherein the one or more metabolites are ATP, AMP, NAD+, ADP, and IMP.

33. A method of providing medical services for a human patient suspected of having or having TBI, said method comprising:
requesting a biological sample from and diagnostic information about the patient, wherein the diagnostic information is a level of one or more metabolites methionine sulfoxide, PC aa C 34:4, ATP, AMP, NAD+, ADP, IMP, spermidine, lysoPC a C20:3, C18:1, and proline in the biological sample; and
administering a therapeutically effective amount of a treatment for TBI when the diagnostic information indicates that the level of the one or more metabolites in the biological sample is at a different level than a statistically validated threshold for the one or more metabolites.

34. The method of claim 33, wherein the treatment is side-lining the patient.

35. The method of claim 34, wherein the subject is side-lined for about 1-2 hours.

36. The method of claim 33, wherein the diagnostic information is determined by Magnetic Resonance spectroscopy (MRS) using a magnetic resonance imaging (MRI) device, nuclear magnetic resonance (NMR), or mass spectrometry (MS) on the biological sample.

37. The method of claim 33, wherein the sample is blood serum.

38. The method of claim 37, wherein the one or more metabolites are methionine sulfoxide and PC aa C 34:4.

39. The method of claim 37, wherein the one or more metabolites are methionine sulfoxide, spermidine, lysoPC a C20:3, C18:1, and proline.

40. The method of claim 33, wherein the one or more metabolites are ATP, AMP, ADP, and IMP.

41. The method of claim 33, wherein the one or more metabolites are AMP, NAD+, ADP, and IMP.

42. The method of claim 33, wherein the one or more metabolites are ATP, AMP, NAD+, ADP, and IMP.

43. A method of monitoring treatment for TBI in a human patient, comprising:
requesting a first biological sample from and first diagnostic information about the patient, wherein the first diagnostic information is a level of one or more metabolites methionine sulfoxide, PC aa C 34:4, ATP, AMP, NAD+, ADP, IMP, spermidine, lysoPC a C20:3, C18:1, and proline in the first biological sample;
administering a therapeutically effective amount of a treatment for TBI to the patient;
after administering the therapeutically effective amount of the treatment for TBI to the patient, requesting a second biological sample from and second diagnostic information about the patient, wherein the second diagnostic information is a level of one or more metabolites methionine sulfoxide, PC aa C 34:4, ATP, AMP, NAD+, ADP, IMP, spermidine, lysoPC a C20:3, C18:1, and proline in the second biological sample; and
comparing the first diagnostic information and the second diagnostic information to determine whether the level of the one or more metabolites in the first biological sample is at a different level than the level of the one or more metabolites in the second biological sample.

44. The method of claim 43, wherein the sample is blood serum.
